(19) United States
(12) Patent Application Publication
(10) Pub. No.: US 2003/0211163 A1
(43) Pub. Date: Nov. 13, 2003

(54) Combination antiviral therapy

(76) Inventor: Kong Teck Chong, Portage, MI (US)

Correspondence Address: Pharmacia & Upjohn Company, Global Intellectual Property, 301 Henrietta Street, Kalamazoo, MI, 49001, US

(21) Appl. No.: 10/339,906

(22) Filed: Jan. 10, 2003

Related U.S. Application Data
(60) Provisional application No. 60/347,550, filed on Jan. 10, 2002.

(51) Int. Cl. A61K 31/7076; A61K 31/7072; A61K 31/54; A61K 31/5377; A61K 31/501; A61K 31/522

(52) U.S. Cl. 424/489; 514/226.5; 514/247; 514/252.01; 514/311; 514/300; 514/456; 514/443; 514/625; 514/45; 514/46; 514/263.38; 514/49; 514/269

(57) ABSTRACT

The invention provides combination therapies for treating papilloma virus.

### CROSS REFERENCE TO RELATED APPLICATIONS

This application claims the benefit of U.S. Serial No. 60/347,550, filed Jan. 10, 2002, under 35 USC 119(e)(i).

### BACKGROUND OF THE INVENTION

Technical Field of the Invention

The present invention relates to a therapy for diseases caused by viruses and more particular to a therapy for treating papilloma virus (PV).

It is estimated that as many as 40 million Americans are infected with PV, and the incidence of this disease appears to be increasing. More than 90 types of PV have been identified by scientists. Papillomavirus is one of the most common causes of sexually transmitted disease (STD) in the United States. In general, there are two kinds of abnormal tissue caused by PV: Condyloma (warts) and Dysplasia (pre-cancer). Condyloma are wart-like growths. They are usually painless, but may cause itching, burning or slight bleeding. Dysplasia is the presence of abnormal cells on the surface of the skin. Dysplasia is not cancer, but may turn into cancer over a period of years if it is not treated.

### SUMMARY OF THE INVENTION

The present invention provides a combination therapy for treating PV. The combination therapy includes administering antiviral agents and inhibitors of cyclooxygenase-2 isozyme (COX-2) to a mammal.

In one aspect, the invention features a pharmaceutical composition including one or more antiviral agent compounds and one or more COX-2 inhibitor compounds. The pharmaceutical composition may include a permeation enhancer. The permeation enhancer may include one or more of the following: ethanol, isopropanol, 1,3-butanediol, oleyl alcohol, thymol, menthol, carvone, carveol, citral, dihydrocarveol, dihydrocarvone, neumenthol, isopulegol, terpene-4-ol, menthone, pulegol, camphor, geraniol, α-terpineol, linalol, carvacrol, t-anethole, and parecoxib.

In another aspect, the invention features a method of treating PV in a mammal by administering a therapeutically effective amount of the combination of one or more antiviral agent compounds and one or more COX-2 inhibitor compounds. The antiviral agent(s) and COX-2 inhibitor(s) may be administered separately or simultaneously. The effective amount of the COX-2 inhibitor and antiviral agent may be administered to the mammal topically.

The above and other aspects, advantages, and novel features of the invention will become apparent from the following detailed description of the invention.

### DESCRIPTION OF THE DRAWINGS

FIG. 1 shows that COX-2 immunoreactivity is localized predominantly to cells within the granular and the spinous layers.

FIG. 2 shows the presence of COX-2 in papillomavirus infected cells and cell grafts obtained from mouse models.

### ABBREVIATIONS AND DEFINITIONS

The term “prevention” includes any of the following: (1) substantially preventing the onset of a clinically evident papillomavirus infection in a subject; (2) preventing the onset of a preclinically evident stage of a papillomavirus infection in a subject; or (3) substantially preventing papillomavirus colonization in a subject. This definition includes prophylactic treatment.

The term “inhibition” as used herein means a decrease in the severity of a papillomavirus infection as compared to that which would occur in the absence of the application of the present invention. This decrease in severity may result from a reduction in viral number, a reduction in viral replication, a reduction in the subject's cell growth infected with the virus, a reduction in cellular replication in the subject, a reduction in cellular mitosis in a subject, a reduction in viral colonization or any combination thereof.

The term “reduced cell growth” is intended to include any reduction in cell growth including the complete cessation of cell growth causing, e.g., apoptosis, in one or more papillomavirus-infected cells.

The phrase “papillomavirus infection” means any presence of a papillomavirus in a subject, irrespective of the stage of infection or colonization.

The phrase “papillomavirus associated disease or related disorder” encompasses any kind of disease or related disorder caused by the virus, including cancers and warts.

The phrase “therapeutically-effective” is intended to qualify the amount of each agent which will achieve the goal of improvement in disorder severity and the frequency of incidence over no treatment or treatment of each agent by itself, while avoiding adverse side effects typically associated with alternative therapies.

The term “subject” for purposes of treatment or prevention includes any human or animal who is susceptible to papillomavirus colonization or infection. The subject can be a domestic livestock species, a laboratory animal species, a zoo animal or a companion animal. In one embodiment, the subject is a mammal. In an alternative embodiment, the mammal is a human being.

The term “cyclooxygenase-2 selective inhibitor” denotes a compound able to inhibit cyclooxygenase-2 without significant inhibition of cyclooxygenase-1. Preferably, it includes compounds that have a cyclooxygenase-2 IC50 of less than about 0.2 micro molar, and also have a selectivity ratio of cyclooxygenase-2 inhibition over cyclooxygenase-1 inhibition of at least 50, and more preferably of at least 100. Even more preferably, the compounds have a cyclooxygenase-1 IC5O of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “hydrido” denotes a single hydrogen atom (H). This hydrido radical may be attached, for example, to an oxygen atom to form a hydroxyl radical or two hydrido radicals may be attached to a carbon atom to form a methylene (—CH2—) radical.

Where used, either alone or within other terms such as “haloalkyl”, “alkylsulfonyl”, “alkoxyalkyl” and “hydroxyalkyl”, the term “alkyl” embraces linear, cyclic or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “halo” means halogens such as fluorine, chlorine, bromine or iodine.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “carbonyl”, whether used alone or with other terms, such as “alkoxycarbonyl”, denotes —(C═O)—.

The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H.

The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl.

The term “alkoxycarbonyl” means a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aralkoxy” embraces aralkyl radicals attached through an oxygen atom to other radicals.

The term “aralkoxyalkyl” embraces aralkoxy radicals attached through an oxygen atom to an alkyl radical.

The term “aralkylthio” embraces aralkyl radicals attached to a sulfur atom.

The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like.

The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “arylamino” denotes amino groups, which have been substituted with one or two aryl radicals, such as N-phenylamino. The “arylamino” radicals may be further substituted on the aryl ring portion of the radical.

The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂.

The term “alkylaminocarbonyl” denotes an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The term “alkylaminoalkyl” embraces radicals having one or more alkyl radicals attached to an aminoalkyl radical.

The term “aryloxyalkyl” embraces radicals having an aryl radical attached to an alkyl radical through a divalent oxygen atom.

The term “arylthioalkyl” embraces radicals having an aryl radical attached to an alkyl radical through a divalent sulfur atom.

### DETAILED DESCRIPTION OF THE INVENTION

A combination therapy for treating PV includes administering to a mammal an antiviral agent and a COX-2 selective inhibitor.

PV refers to a pre-cancerous condition. More than 90 different types of PV have been classified. These include both “cutaneous” and “mucosal” PV types. In general, cutaneous types infect keratinizing epithelium, and are responsible for causing various skin warts. The mucosal types infect non-keratinizing epithelium including the oral mucosa, conjunctiva, respiratory tract, and the anogenital area. Several types, including PV 6, 11 and 42, are associated with raised, rough, easily visible genital warts Other types are associated with flat warts. More importantly, certain types are associated with pre-malignant and malignant changes in the cervix (abnormal Papanicolaou or Pap smears). These include types 16, 18, 31, 33, 35, 39, 45, 51, and 52. Genital tract PV infection is thought to be the most common sexually transmitted disease (STD) in the United States. Infection of the genital and anal regions with PV can cause warts (anogenital condyloma) on the penis, vulva, urethra, vagina, cervix, and around the anus. Lesions on the external genitalia are easily recognized. On the penis, genital warts tend to be drier and more limited than on the female genitalia or around the anus of either sex. They are raised, rough, flesh-colored “warty” appearing lesions that may occur singly or in clusters. Warts around the anus and vulva may rapidly enlarge, taking on a “cauliflower-like” appearance. In women, a pelvic examination may reveal growths on the vaginal walls or the cervix by a procedure called colposcopy. The tissue of the vagina and cervix may be treated with acetic acid to make flat warts visible. A better way to detect and diagnose PV disease is by performing a PAP test, which involve the microscopic examination of exfoliated cell samples in cervical smears. The appearance of abnormal cells on the surface of the cervix is described as cervical dysplasia. Dysplasia is considered to be a precancerous condition. Left untreated, dysplasia sometimes progresses to an early form of cancer known as cervical carcinoma in situ, and eventually to invasive cervical cancer. In addition to the PAP test, more modem approach involves the detection and typing of PV DNA. This can be done by various techniques, including DNA hybridization with or without prior amplification (PCR) of the target PV DNA.

PV associated warts and dysplasia can be differentiated from cancerous conditions by the staging of disease using the Bethesda System (National Cancer Institute) or the CIN Grading System (Sherman ME, 2001. Critical view on morphological methods to assess PV infections, Abstract, pages 54-55, 19^{th} International Papillomavirus Conference). The Bethesda System was developed by the CDC and NIH in order to have a comprehensive and standardized method of classifying Pap smear results. It uses the term squamous intraepithelial lesion (SIL) to describe abnormal changes in the cells on the surface of the cervix. Squamous refers to thin, flat cells that lie on the outer surface of the cervix. An intraepithelial lesion occurs when a layer of abnormal cells replaces normal cells on the cervical surface, and these changes are classified as high grade or low grade. The CIN Grading System uses the term cervical intraepithelial neoplasia (CIN) to describe new abnormal growth of cells on the surface layers of the cervix. The CIN System grades the degree of cell abnormality numerically, with CIN 1 being the lowest and CIN 3 being the highest. The wart and pre-cancerous stages of the PV lesions include both low and high grade SIL as defined by the Bethesda system, or CIN 1 to CIN 3 by the CIN Grading or WHO System. A summary of these grading system is as shown in the table below.

Nomenclature in Cervical Cytology

| PAP system | WHO system | Bethesda system |
|---|---|---|
| Class I | Normal | Within normal limits |
| Class II | Inflammatory atypia | Infection |
| | | Reactive or reparative changes |
| Class IIR | Squamous atypia | Squamous cell abnormalities |
| | PV atypia | Atypical squamous cells |
| | | of undetermined significance |
| | | Low grade |
| Class III | Dysplasia | Squamous intraepithelial |
| | | lesion |
| | Mild (CIN 1) | Low grade |
| | Moderate (CIN 2) | High grade |
| | Severe (CIN 3) | High grade |
| Class IV | Carcinoma in situ (CIN 3) | High grade |
| Class V | Invasive SCCA | Squamous cell carcinoma |
| | | Galndular cell abnormalities; |
| | Adenocarcinoma | Adenocarcinoma |
| | | Nonepithelial malignant |
| | | neoplasm |

The term “antiviral agent” refers to a compound that exhibits activity against diseases caused by viruses. Certain antiviral agents, such as antimitotic agents, exhibits activity against diseases caused by viruses by inhibiting or preventing mitosis or nuclear division of the subject's cell. Generally speaking, these agents slow viral replication and concomitantly, viral growth, by preventing division of a subject's cells infected with PV. In addition to slowing viral replication and growth, these agents also advantageously cause lesions resulting from viral infection to substantially reduce in size.

Examples of suitable antiviral agents include, but are not limited to, Podophyllin (Podophyllotoxins); Nucleoside analoques; Immunomodulators (interferons, imiquimod, cytokines); Antisense oligonucleotides; Prophylactic vaccines and therapeutic vaccines; and non-nucleoside inhibitors. Antiviral agents may be obtained commercially or be prepared according to the references cited in PHYSICIANS' DESK REFERENCE, the 54^{th} Edition (2000) and the US FDA's Orange book. Additional antiviral agents may be found, for example, the PHYSICIANS' DESK REFERENCE, the MERCK Manual or the MERCK Index.

Podophyllin (Podophyllotoxins) are chemical cell replication blockers, such as podofilox or podophyllin, and are generally used in treatments for removing existing warts. Chemical cell replication blockers typically offer temporary symptomatic relief when administered alone. Pharmaceutical compositions including a COX-2 inhibitor and a chemical cell replication blocker provide prolonged symptomatic relief. Podophyllotoxin selectively arrests mitosis in the metaphase stage of infected cutaneous cells, causing necrosis of the infected cells. The ability to selectively arrest mitosis at this particular stage is highly advantageous because it leads directly to removal of the lesion caused by the papillomavirus. The podophyllotoxin may be obtained from a number of sources. For example, in one embodiment, the podophyllotoxin may be obtained from a number of commercially available sources sold under tradenames such as podofilox (brand name “Condylox®” supplied by Oclassen Pharmaceuticals, Inc.), which is a glucoside extract synthesized chemically or purified from the plant families Coniferae and Berberidaceae. In yet another embodiment, the podophyllotoxin may be obtained from podphyllum resin (brand name “Pod-Ben-25” or “Podofin®”), which is a powdered mixture of resins removed from Podophyllum peltatum (more commonly known as the mayapple or American mandrake), a pereninial plant in the Berberidaceae family and found in the woodlands in Canada and the Eastern United States. Both agents are particularly suitable for removing certain types of warts on the outside of the skin of the genital area, including condyloma acuminata (commonly known as ano-genital warts) because they are not caustic to the skin.

Other antimitotic agents are oxygenated esters of 4-idodophenylamino benzhydroxamic acid or derivatives thereof as disclosed in WO/00206213, which is hereby incorporated by reference in its entirety. These agents inhibit MAP kinase, which is an enzyme essential for cellular proliferation. Inhibition of this enzyme completely arrests mitogenesis. Methods and modes of administration of these agents can be found in WO/00206213.

Nucleoside analoques target virus polymerases and represents the majority of the specific antiviral drugs currently in use. The majority of these drugs function as polymerase substrate (i.e. nucleoside/nucleotide) analogues. Examples of nucleoside analogues that have been shown to inhibit members of the herpevirus family are acyclovir, penciclovir, famciclovir, ganciclovir, BVDU, broavir, HPMPA, FIAC, FIAU, and Cidofovir (HPMPC). Other nucleoside analogs including Zidovudine (AZT), Zalcitabine (ddC), Didanosine (ddI), Lamivudine (3TC) and Stavudine (d4T) have been shown to be active against HIV infection. Examples of the nucleoside analogs that have been shown to be active against PV are vidarabine, HPMPC, and ribavirin. Vidarabine, a DNA polymerase inhibitor, suppresses growth and PV gene expression in human cervical keratinocytes immortalized by PV or in cervical cancer cell lines. Ribavirin (triazole carboxamide) inhibits replication of many DNA and RNA viruses. Cidofovir (HPMPC) inhibits a broad range of DNA viruses. Some chemical agents have been shown to have a broad spectrum of activity against many different viruses. For example foscarnet (PFA, trisodium phosphonoformate) is a non-nucleoside inhibitor that blocks the function of the DNA and RNA polymerases of many DNA and RNA viruses.

In yet a further aspect of the invention, the antiviral agent is an antineoplastic agent. These agents reduce cell proliferation and thus arrest the growth of new cells or tissue, which may be benign or malignant. Although historically employed as a chemotherapeutic agent, antineoplastic agents are highly effect against a broad spectrum of papillomavirus. In one embodiment, the antineoplastic agent is 5-fluorouracil. 5-Fluorouracil (Efudex®, Adrucil®, Fluoroplex®) interferes with DNA synthesis by blocking the methylation of deoxyuridylic acid and inhibits thymidylate syntheses, which subsequently reduces cell proliferation. In another embodiment, the antineoplastic agent is an oxygenated ester of 4-iodophenylamino benzhydroxamic acid. These compounds are further described in WO/0206213, which is hereby incorporated by reference in its entirety. In yet another alternative of this embodiment, the antineoplastic agent is bleomycin (brand name “Blenoxane®”). A further aspect of the invention encompasses anti-papillomavirus agents that are desiccant agents. Desiccants dehydrate lesions caused by the papillomavirus. After several days to a few weeks of treatment with a desiccant, the lesion eventually dries and can be easily removed. By way of example, in one embodiment the desiccant agent is Tricholoracetic acid (TCA). TCA is a highly corrosive desiccating agent that cauterizes skin, keratin, and other tissues and is commercially available as Tri-Chlor.

Several non-nucleoside inhibitors have been approved for the treatment of HIV infection (Buckheit RW. 2001. Non-nucleoside reverse transcriptase inhibitors: perspectives on novel therapeutic compounds and strategies for the treatment of HIV infection. Expert Opinion on Investigational Drugs. 10(8): 1423-1442. Some examples are nevirapine (Viramune(TM) Boehringer Ingelheim), delavirdine (Rescriptor(TM): Pharmacia and Upjohn) and efavirenz (Sustiva(TM): Dupont Pharmaceuticals). For PV infection, there is no currently available non-nucleoside inhibitor, but several compounds with activities against PV are in development (Hajduk P J. Dinges J. Miknis G F. Merlock M. Middleton T. Kempf D J. Egan D A. Walter K A. Robins T S. Shuker S B. Holzman T F. Fesik S W. 1997. NMR-based discovery of lead inhibitors that Block DNA binding of the human papillomavirus E2 protein. Journal of Medicinal Chemistry. 40:3144-3150).

Immunomodulators or immune response modifiers (interferons, imiquimod, cytokines) are agents that have no direct effect on the virus or viral replication mechanism, but are able to enhance host defense against infection. Generally speaking, Immunomodulators or immune response modifiers allow the body to rid itself of the virus by substantially increasing the immune response of the subject. Examples include various interferons, cytokines, and small molecules that influence the production of interferons and cytokines. Imiquimod is a synthetic molecule with immune-modulating properties that activate monocytes/macrophages via binding to cell surfaces receptors resulting in the secretion of interferon-alpha and other proinflammatory cytokines including TNF-alpha, IL-12.

The interferon employed as an antiviral agent is a recombinant protein. Recombinant interferon may be obtained from a number of sources. For example, in one embodiment, the interferon is interferon alfa-2a (Roferon®-A), interferon alfa-2d (Intron® A supplied by Shering Corp.) or interferon beta-1b (Betaseron®), all three of which may be produced by recombinant DNA technology that employs a genetically engineered Escherichia coli bacterium containing DNA that codes for the human protein. In another embodiment, the recombinant interferon is interferon gamma-1b (Actimmune®), which activates the immune system by stimulating a class of immune cells known as macrophages.

In yet another embodiment, the interferon employed is a naturally occurring protein purified from any suitable source. By way of example, a native interferon suitable for use in the current invention is interferon alfa-n3 (Alferon N®). Alferon N®, which is partially glycosylated, synthesized by and purified from human white blood cells. Further, Alferon N® is particularly suitable for use in the present invention as it exists in many different isoforms and therefore, it has broad-spectrum activity against a wide variety of papillomavirus types.

It is also contemplated that immune stimulants other than interferons may be used as antiviral agents in the practice of the invention. In one such embodiment, the immune stimulant is imiquimod. Imiquimod (brand name “Aldara®”) is an immune response modifier that stimulates the immune system to release a number cytokines that mediate numerous immune responses. In particular, imiquimod causes the release of numerous cytokines that substantially inhibit replication of the papillomavirus.

In yet another embodiment, the immune stimulant is cimetidine. Cimetidine, commonly known as “Tagamet®”, is a histamine H2-receptor antagonist. This agent inhibits H2 receptors found on suppressor T cells. H2-receptors signal the body to secrete histamine, which in turn, inhibits an immune response. Accordingly, by inhibiting suppressor T cells, cimetidine stimulates the immune system to build up a more effective response against papillomavirus infection.

Antisense oligonucleotides are short synthetic oligonucleotides having complementary sequences to viral mRNA that have been shown to inhibit viral gene expression. By masking part of the corresponding RNA template with a custom-designed DNA fragment able to bind firmly to the selected RNA sequence, an antisense inhibitor can halt the the production of specific viral proteins. An example of an antisense drug is fomivirsen, which is used to treat eye infections caused by cytomegalovirus in AIDS patients. Antisense inhibitors have been reported for human papillomavirus. See, for example, Antisense & Nucleic Acid Drug Development. 9(5): 441-450 (1999); and Proceedings of the National Academy of Sciences of the United States of America. 95(3): 1189-1194 (1998), both authored by Alvarez-Salas et al.

Prophylactic vaccines are designed to boost the production of antibodies that prevent the establishment of papillomavirus infection. Therapeutic vaccines boost the cytotoxic T-cell response. These vaccines when administered in combination with COX-2 inhibitors reduce target cell population of PV infections.

The PV therapy includes administering a therapeutically effective amount of one or more COX-2 inhibitors or pharmaceutically acceptable salts thereof to a mammal.

The terms “cyclooxygenase-2 selective inhibitor,” “COX-2 selective inhibitor,” and COX-2 inhibitor interchangeably refer to a therapeutic compound which selectively inhibits the COX-2 isoform of the enzyme cyclooxygenase. In practice, COX-2 selectivity varies depending on the conditions under which the test is performed and on the inhibitors being tested. However, for the purposes of this patent, COX-2 selectivity can be measured as a ratio of the in vitro or in vivo IC₅₀ value for inhibition of COX-1, divided by the IC₅₀ value for inhibition of COX-2. A COX-2 selective inhibitor is any inhibitor for which the ratio of COX-1 IC₅₀ to COX-2 IC₅₀ is greater than 1, preferably greater than 5, more preferably greater than 10, still more preferably greater than 50, and more preferably still greater than 100.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

Cyclooxygenase Inhibitors

The present invention discloses that treatment of a subject with one or more cyclooxygenase inhibitors results in the effective treatment of PV relative to previously disclosed treatment regimens. The method comprises treating the subject with an amount a cyclooxygenase inhibitor or acceptable salt or derivative or prodrug, in which the amount of the cyclooxygenase inhibitor constitutes a PV-condition effective amount of the cyclooxygenase inhibitor.

In one embodiment of the invention the COX-2 selective inhibitor is meloxicam, Formula A-1 (CAS registry number 71125-38-7) or a pharmaceutically acceptable salt or derivative or prodrug thereof.

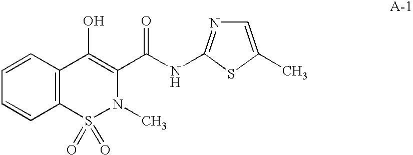

In another embodiment of the invention the cyclooxygenase-2 selective inhibitor is the COX-2 selective inhibitor RS-57067, 6-[[5-(4-chlorobenzoyl)-1,4-dimethyl-1H-pyrrol-2-yl]methyl]-3(2H)-pyridazinone, Formula A-2 (CAS registry number 179382-91-3) or a pharmaceutically acceptable salt or derivative or prodrug thereof.

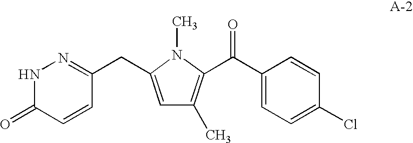

In another embodiment of the invention the cyclooxygenase-2 selective inhibitor is the COX-2 selective inhibitor ABT-963, 2-(3,4-difluorophenyl)-4-(3-hydroxy-3-methylbutoxy)-5-[4-(methylsulfonyl)phenyl]-(9C1)-3(2H)-pyridazinone, Formula A-3 (CAS registry number 266320-83-6 or a pharmaceutically acceptable salt or derivative or prodrug thereof.

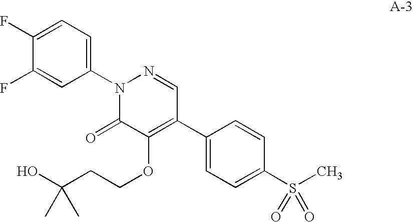

In another embodiment of the invention the cyclooxygenase-2 selective inhibitor is the COX-2 selective inhibitor COX-189, Formula A-4 (CAS registry number 346670-74-4) or a pharmaceutically acceptable salt or derivative or prodrug thereof.

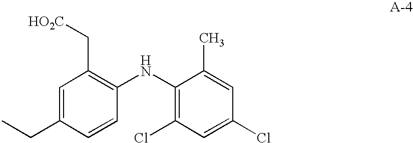

In another embodiment of the invention the cyclooxygenase-2 selective inhibitor is the COX-2 selective inhibitor NS-398, N-(2-cyclohexyl-4-nitrophenyl)methanesulfonamide, Formula A-5 (CAS registry number 123653-11-2) or a pharmaceutically acceptable salt or derivative or prodrug thereof.

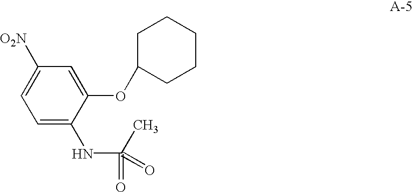

In a preferred embodiment of the invention the cyclooxygenase-2 selective inhibitor is a COX-2 selective inhibitor of the chromene structural class. For the purposes of the present invention a chromene class COX-2 selective inhibitor is a substituted benzopyran or a substituted benzopyran compound selected from the group consisting of substituted a benzothiopyran, a dihydroquinoline, or a dihydronaphthalene having the general Formula II shown below. Some chromene compounds useful as COX-2 selective inhibitors in the present invention are shown in Table 3, including the diastereomers, enantiomers, racemates, tautomers, salts, esters, amides and prodrugs thereof.

TABLE 3
Examples of Chromene COX-2 Selective
Inhibitors as Embodiments

| | |
|---|---|
| | II |
| 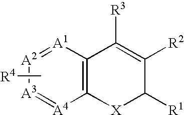 | |

| Compound | Structural |
|---|---|
| Number | Formula |
| A-6 | 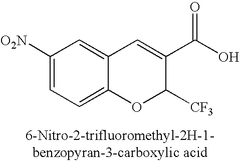 |
| A-7 | 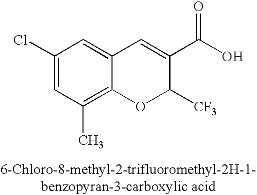 |
| A-8 | 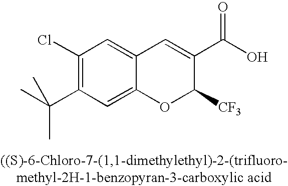 |
| A-9 | 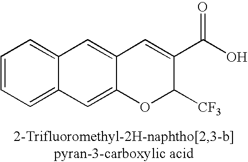 |
| A-10 | 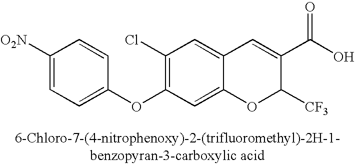 |
| A-11 | 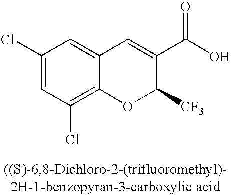 |
| A-12 | 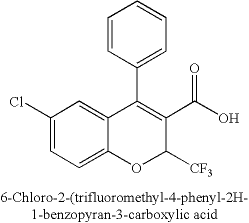 |
| A-13 | 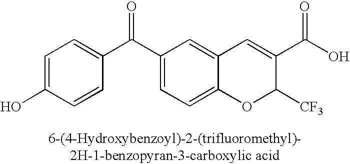 |
| A-14 | 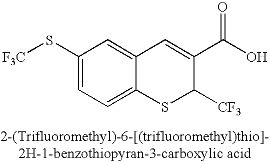 |
| A-15 | 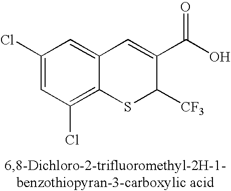 |
| A-16 | 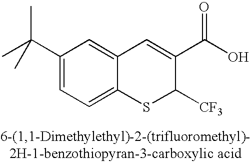 |
| A-17 | 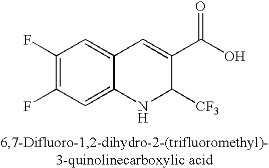 |
| A-18 | 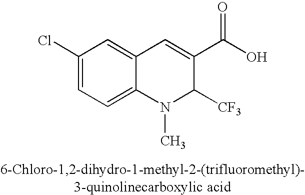 |
| A-19 | 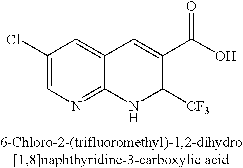 |
| A-20 | 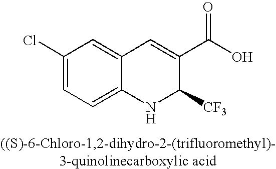 |

The individual patent documents referenced in Table 4 below describe the preparation of the COX-2 inhibitors of Table 3 and the patent documents are each herein incorporated by reference.

TABLE 4
References for Preparation of Chromene
COX-2 Inhibitors

| Compound Number | Patent Reference |
|---|---|
| A-6 | U.S. Pat. No. 6,077,850; example 37 |
| A-7 | U.S. Pat. No. 6,077,850; example 38 |
| A-8 | U.S. Pat. No. 6,077,850; example 68 |
| A-9 | U.S. Pat. No. 6,034,256; example 64 |
| A-10 | U.S. Pat. No. 6,077,850; example 203 |
| A-11 | U.S. Pat. No. 6,034,256; example 175 |
| A-12 | U.S. Pat. No. 6,077,850; example 143 |
| A-13 | U.S. Pat. No. 6,077,850; example 98 |
| A-14 | U.S. Pat. No. 6,077,850; example 155 |
| A-15 | U.S. Pat. No. 6,077,850; example 156 |
| A-16 | U.S. Pat. No. 6,077,850; example 147 |
| A-17 | U.S. Pat. No. 6,077,850; example 159 |
| A-18 | U.S. Pat. No. 6,034,256; example 165 |
| A-19 | U.S. Pat. No. 6,077,850; example 174 |
| A-20 | U.S. Pat. No. 6,034,256; example 172 |

In a further preferred embodiment of the invention the cyclooxygenase inhibitor is selected from the class of tricyclic cyclooxygenase-2 selective inhibitors represented by the general structure of Formula III

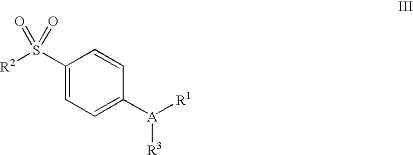

wherein A is a substituent selected from partially unsaturated or unsaturated heterocyclyl and partially unsaturated or unsaturated carbocyclic rings;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl or amino; and

wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt or derivative or prodrug thereof.

In a still more preferred embodiment of the invention the cyclooxygenase-2 selective inhibitor represented by the above Formula III is selected from the group of compounds, illustrated in Table 5, consisting of celecoxib (A-21), valdecoxib (A-22), deracoxib (A-23), rofecoxib (A-24), etoricoxib (MK-663; A-25), JTE-522 (A-26), parecoxib (A-27), or a pharmaceutically acceptable salt or derivative or prodrug thereof.

In an even more preferred embodiment of the invention the COX-2 selective inhibitor is selected from the group consisting of celecoxib, rofecoxib and etoricoxib.

TABLE 5
Examples of Tricyclic COX-2 Selective
Inhibitors as Embodiments

| Compound | Structural |
|---|---|
| Number | Formula |
| A-21 | 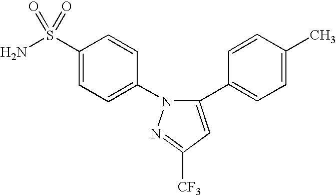 |
| A-22 | 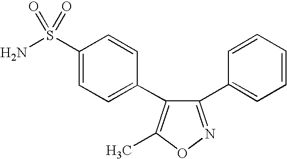 |
| A-23 | 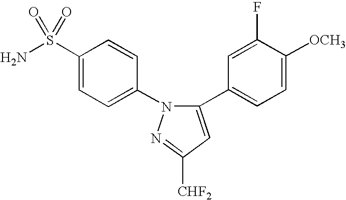 |
| A-24 | 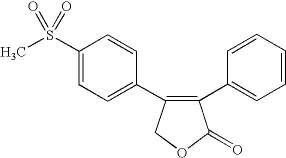 |
| A-25 | 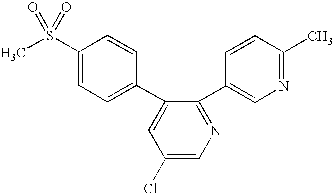 |
| A-26 | 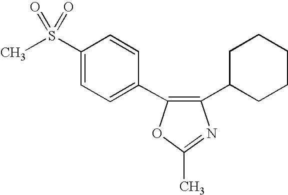 |

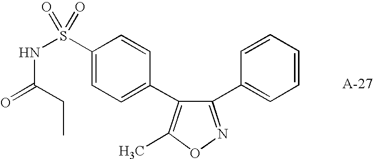

Parecoxib (A-27, U.S. Pat. No. 5,932,598, CAS No. 198470-84-7), which is a therapeutically effective prodrug of the tricyclic cyclooxygenase-2 selective inhibitor valdecoxib, A-22, may be advantageously employed as a source of a COX-2 inhibitor (U.S. Pat. No. 5,932,598, herein incorporated by reference).

The individual patent documents referenced in Table 6 below describe the preparation of the aforementioned cyclooxygenase-2 selective inhibitors A-21 through A-27 and are each herein incorporated by reference.

TABLE 6
References for Preparation of Tricyclic
COX-2 Inhibitors and Prodrugs

| Compound Number | Patent Reference |
|---|---|
| A-21 | U.S. Pat. No. 5,466,823 |
| A-22 | U.S. Pat. No. 5,633,272 |
| A-23 | U.S. Pat. No. 5,521,207 |
| A-24 | U.S. Pat. No. 5,840,924 |
| A-25 | WO 98/03484 |
| A-26 | WO 00/25779 |
| A-27 | U.S. Pat. No. 5,932,598 |

U.S. Pat. No. 6,180,651 describes COX-2 selective inhibitors of the diarylmethylidene furan derivative which are useful in the combination of the present invention. In a preferred embodiment of the present invention, the diarylmethylidene furan derivative COX-2 inhibitor is BMS-347070.

Other COX-2 inhibitors are described below.

The compound having the formula B-25 that has been previously described in International Publication number WO 00/24719 (which is herein incorporated by reference), is another tricyclic cyclooxygenase-2 selective inhibitor which may be advantageously employed.

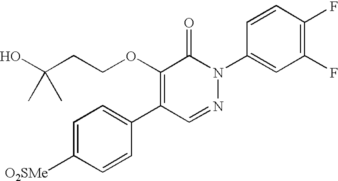

Another cyclooxygenase-2 selective inhibitor that is useful in connection with the method(s) of the present invention is N-(2-cyclohexyloxynitrophenyl)-methane sulfonamide (NS-398) having a structure shown below as B-26.

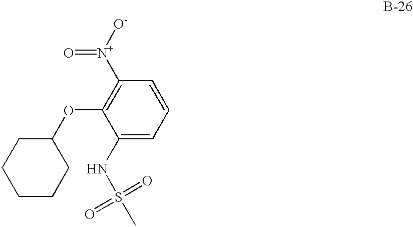

In yet a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula IIIa:

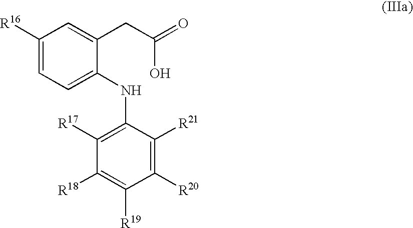

or an isomer, a pharmaceutically acceptable salt, ester, or prodrug thereof;

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula IIIa or an isomer, a pharmaceutically acceptable salt, ester, or prodrug thereof, wherein:

R¹⁶ is ethyl;

R¹⁷ and R¹⁹ are chloro;

R¹⁸ and R²⁰ are hydrogen; and

and R²¹ is methyl.

In yet another embodiment, the cyclooxygenase-2 selective inhibitor is represented by Formula (IV):

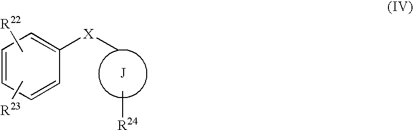

or an isomer, a pharmaceutically acceptable salt, an ester, or a prodrug thereof,

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V):

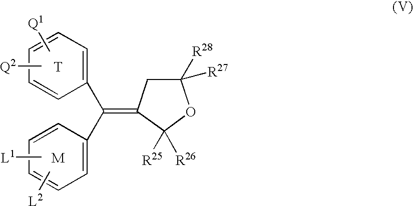

or an isomer, a pharmaceutically acceptable salt, an ester, or a prodrug thereof,

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In a particularly preferred embodiment, the compounds N-(2-cyclohexyloxynitrophenyl)methane sulfonamide, and (E)-4-[(4-methylphenyl)(tetrahydro-2-oxo-3-furanylidene) methyl] benzenesulfonamide having the structure of Formula (V) are employed as cyclooxygenase-2 selective inhibitors.

Exemplary compounds that are useful for the cyclooxygenase-2 selective inhibitor in connection with the method(s) of the present invention, the structures for which are set forth in Table 3 below, include, but are not limited to: 6-chloro-2-trifluoromethyl-2H-1-benzopyran-3-carboxylic acid;

6-chloro-7-methyl-2-trifluoromethyl-2H-1-benzopyran-3-carboxylic acid;

8-(1-methylethyl)-2-trifluoromethyl-2H-1-benzopyran-3-carboxylic acid;

6-chloro-8-(1-methylethyl)-2-trifluoromethyl-2H-1-benzopyran-3-carboxylic acid;

2-trifluoromethyl-3H-naphtho[2,1-b]pyran-3-carboxylic acid;

7-(1,1-dimethylethyl)-2-trifluoromethyl-2H-1-benzopyran-3-carboxylic acid;

6-bromo-2-trifluoromethyl-2H-1-benzopyran-3-carboxylic acid;

8-chloro-2-trifluoromethyl-2H-1-benzopyran-3-carboxylic acid;

6-trifluoromethoxy-2-trifluoromethyl-2H-1-benzopyran-3-carboxylic acid;

5,7-dichloro-2-trifluoromethyl-2H-1-benzopyran-3-carboxylic acid;

8-phenyl-2-trifluoromethyl-2H-1-benzopyran-3-carboxylic acid;

7,8-dimethyl-2-trifluoromethyl-2H-1-benzopyran-3-carboxylic acid;

6,8-bis(dimethylethyl)-2-trifluoromethyl-2H-1-benzopyran-3-carboxylic acid;

7-(1-methylethyl)-2-trifluoromethyl-2H-1-benzopyran-3-carboxylic acid;

7-phenyl-2-trifluoromethyl-2H-1-benzopyran-3-carboxylic acid;

6-chloro-7-ethyl-2-trifluoromethyl-2H-1-benzopyran-3-carboxylic acid;

6-chloro-8-ethyl-2-trifluoromethyl-2H-1-benzopyran-3-carboxylic acid;

6-chloro-7-phenyl-2-trifluoromethyl-2H-1-benzopyran-3-carboxylic acid;

6,7-dichloro-2-trifluoromethyl-2H-1-benzopyran-3-carboxylic acid;

6,8-dichloro-2-trifluoromethyl-2H-1-benzopyran-3-carboxylic acid;

6-chloro-8-methyl-2-trifluoromethyl-2H-1-benzopyran-3-carboxylic acid;

8-chloro-6-methyl-2-trifluoromethyl-2H-1-benzopyran-3-carboxylic acid;

8-chloro-6-methoxy-2-trifluoromethyl-2H-1-benzopyran-3-carboxylic acid;

6-bromo-8-chloro-2-trifluoromethyl-2H-1-benzopyran-3-carboxylic acid;

8-bromo-6-fluoro-2-trifluoromethyl-2H-1-benzopyran-3-carboxylic acid;

8-bromo-6-methyl-2-trifluoromethyl-2H-1-benzopyran-3-carboxylic acid;

8-bromo-5-fluoro-2-trifluoromethyl-2H-1-benzopyran-3-carboxylic acid;

6-chloro-8-fluoro-2-trifluoromethyl-2H-1-benzopyran-3-carboxylic acid;

6-bromo-8-methoxy-2-trifluoromethyl-2H-1-benzopyran-3-carboxylic acid;

6-[[(phenylmethyl)amino]sulfonyl]-2-trifluoromethyl-2H-1-benzopyran-3-carboxylic acid;

6-[(dimethylamino)sulfonyl]-2-trifluoromethyl-2H-1-benzopyran-3-carboxylic acid;

6-[(methylamino)sulfonyl]-2-trifluoromethyl-2H-1-benzopyran-3-carboxylic acid;

6-[(4-morpholino)sulfonyl]-2-trifluoromethyl-2H-1-benzopyran-3-carboxylic acid;

6-[(1, 1-dimethylethyl)aminosulfonyl]-2-trifluoromethyl-2H-1-benzopyran-3-carboxylic acid;

6-[(2-methylpropyl)aminosulfonyl]-2-trifluoromethyl-2H-1-benzopyran-3-carboxylic acid;

6-methylsulfonyl-2-trifluoromethyl-2H-1-benzopyran-3-carboxylic acid;

8-chloro-6-[[(phenylmethyl)amino]sulfonyl]-2-trifluoromethyl-2H-1-benzopyran-3-carboxylic acid;

6-phenylacetyl-2-trifluoromethyl-2H-1-benzopyran-3-carboxylic acid;

6,8-dibromo-2-trifluoromethyl-2H-1-benzopyran-3-carboxylic acid;

8-chloro-5,6-dimethyl-2-trifluoromethyl-2H-1-benzopyran-3-carboxylic acid;

6,8-dichloro-(S)-2-trifluoromethyl-2H-1-benzopyran-3-carboxylic acid;

6-benzylsulfonyl-2-trifluoromethyl-2H-1-benzopyran-3-carboxylic acid;

6-[[N-(2-furylmethyl)amino] sulfonyl]-2-trifluoromethyl-2H-1-benzopyran-3-carboxylic acid;

6-[[N-(2-phenylethyl)amino]sulfonyl]-2-trifluoromethyl-2H-1-benzopyran-3-carboxylic acid;

6-iodo-2-trifluoromethyl-2H-1-benzopyran-3-carboxylic acid;

7-(1,1-dimethylethyl)-2-pentafluoroethyl-2H-1-benzopyran-3-carboxylic acid;

6-chloro-2-trifluoromethyl-2H-1-benzothiopyran-3-carboxylic acid;

3-[(3-Chloro-phenyl)-(4-methanesulfonyl-phenyl)-methylene]-dihydro-furan-2-one;

8-acetyl-3-(4-fluorophenyl)-2-(4-methylsulfonyl)phenyl-imidazo(1,2-a)pyridine;

5,5-dimethyl-4-(4-methylsulfonyl)phenyl-3-phenyl-2-(5H)-furanone;

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)pyrazole;

4-(5-(4-chlorophenyl)-3-(4-methoxyphenyl)-1H-pyrazol-1-yl)benzenesulfonamide;

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide;

4-[5-(4-chlorophenyl)-3-(trifluoromethyl)-1H-pyrazol-1-yl]benzenesulfonamide;

4-[5-phenyl-3-(trifluoromethyl)-1H-pyrazol-1-yl]benzenesulfonamide;

4-[5-(4-fluorophenyl)-3-(trifluoromethyl)-1H-pyrazol-1-yl]benzenesulfonamide;

4-[5-(4-methoxyphenyl)-3-(trifluoromethyl)-1H-pyrazol-1-yl]benzenesulfonamide;

4-[5-(4-chlorophenyl)-3-(difluoromethyl)-1H-pyrazol-1-yl]benzenesulfonamide;

4-[5-(4-methylphenyl)-3-(trifluoromethyl)-1H-pyrazol-1-yl]benzenesulfonamide;

4-[4-chloro-5-(4-chlorophenyl)-3-(trifluoromethyl)-1H-pyrazol-1-yl]benzenesulfonamide;

4-[3-(difluoromethyl)-5-(4-methylphenyl)-1H-pyrazol-1-yl]benzenesulfonamide;

4-[3-(difluoromethyl)-5-phenyl-1H-pyrazol-1-yl]benzenesulfonamide;

4-[3-(difluoromethyl)-5-(4-methoxyphenyl)-1H-pyrazol-1-yl]benzenesulfonamide;

4-[3-cyano-5-(4-fluorophenyl)-1H-pyrazol-1-yl]benzenesulfonamide;

4-[3-(difluoromethyl)-5-(3-fluoro-4-methoxyphenyl)-1H-pyrazol-1-yl]benzenesulfonamide;

4-[5-(3-fluoro-4-methoxyphenyl)-3-(trifluoromethyl)-1H-pyrazol-1-yl]benzenesulfonamide;

4-[4-chloro-5-phenyl-1H-pyrazol-1-yl]benzenesulfonamide;

4-[5-(4-chlorophenyl)-3-(hydroxymethyl)-1H-pyrazol-1-yl]benzenesulfonamide;

4-[5-(4-(N,N-dimethylamino)phenyl)-3-(trifluoromethyl)-1H-pyrazol-1-yl]benzenesulfonamide;

5-(4-fluorophenyl)-6-[4-(methylsulfonyl)phenyl] spiro[2.4]hept-5-ene;

4-[6-(4-fluorophenyl)spiro[2.4]hept-5-en-5-yl]benzenesulfonamide;

6-(4-fluorophenyl)-7-[4-(methylsulfonyl)phenyl] spiro[3.4]oct-6-ene;

5-(3-chloro-4-methoxyphenyl)-6-[4-(methylsulfonyl)phenyl]spiro[2.4]hept-5-ene;

4-[6-(3-chloro-4-methoxyphenyl)spiro[2.4]hept-5-en-5-yl]benzenesulfonamide;

5-(3,5-dichloro-4-methoxyphenyl)-6-[4-(methylsulfonyl)phenyl] spiro[2.4]hept-5-ene;

5-(3-chloro-4-fluorophenyl)-6-[4-(methylsulfonyl)phenyl]spiro[2.4]hept-5-ene;

4-[6-(3,4-dichlorophenyl)spiro[2.4]hept-5-en-5-yl]benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluoromethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzylaminothiazole;

4-(4-fluorophenyl)-5-(4-methylsulfonylphenyl)-2-(1-propylamino)thiazole;

2-[(3,5-dichlorophenoxy)methyl)-4-(4-fluorophenyl)-5-[4-(methylsulfonyl)phenyl]thiazole;

5-(4-fluorophenyl)-4-(4-methylsulfonylphenyl)-2-trifluoromethylthiazole;

1-methylsulfonyl-4-[1, 1-dimethyl-4-(4-fluorophenyl)cyclopenta-2,4-dien-3-yl]benzene;

4-[4-(4-fluorophenyl)-1, 1-dimethylcyclopenta-2,4-dien-3-yl]benzenesulfonamide;

5-(4-fluorophenyl)-6-[4-(methylsulfonyl)phenyl]spiro[2.4]hepta-4,6-diene;

4-[6-(4-fluorophenyl)spiro[2.4]hepta-4,6-dien-5-yl]benzenesulfonamide;

6-(4-fluorophenyl)-2-methoxy-5-[4-(methylsulfonyl)phenyl]-pyridine-3-carbonitrile;

2-bromo-6-(4-fluorophenyl)-5-[4-(methylsulfonyl)phenyl]-pyridine-3-carbonitrile;

6-(4-fluorophenyl)-5-[4-(methylsulfonyl)phenyl]-2-phenyl-pyridine-3-carbonitrile;

4-[2-(4-methylpyridin-2-yl)-4-(trifluoromethyl)-1H-imidazol-1-yl]benzenesulfonamide;

4-[2-(5-methylpyridin-3-yl)-4-(trifluoromethyl)-1H-imidazol-1-yl]benzenesulfonamide;

4-[2-(2-methylpyridin-3-yl)-4-(trifluoromethyl)-1H-imidazol-1-yl]benzenesulfonamide;

3-[1-[4-(methylsulfonyl)phenyl]-4-(trifluoromethyl)-1H-imidazol-2-yl]pyridine;

2-[1-[4-(methylsulfonyl)phenyl-4-(trifluoromethyl)-1H-imidazol-2-yl]pyridine;

2-methyl-4-[1-[4-(methylsulfonyl)phenyl-4-(trifluoromethyl)-1H-imidazol-2-yl]pyridine;

2-methyl-6-[1-[4-(methylsulfonyl)phenyl-4-(trifluoromethyl)-1H-imidazol-2-yl]pyridine;

4-[2-(6-methylpyridin-3-yl)-4-(trifluoromethyl)-1H-imidazol-1-yl]benzenesulfonamide;

2-(3,4-difluorophenyl)-1-[4-(methylsulfonyl)phenyl]-4-(trifluoromethyl)-1H-imidazole;

4-[2-(4-methylphenyl)-4-(trifluoromethyl)-1H-imidazol-1-yl]benzenesulfonamide;

2-(4-chlorophenyl)-1-[4-(methylsulfonyl)phenyl]-4-methyl-1H-imidazole;

2-(4-chlorophenyl)-1-[4-(methylsulfonyl)phenyl]-4-phenyl-1H-imidazole;

2-(4-chlorophenyl)-4-(4-fluorophenyl)-1-[4-(methylsulfonyl)phenyl]-1H-imidazole;

2-(3-fluoro-4-methoxyphenyl)-1-[4-(methylsulfonyl)phenyl-4-(trifluoromethyl)-1H-imidazole;

1-[4-(methylsulfonyl)phenyl]-2-phenyl-4-trifluoromethyl-1H-imidazole;

2-(4-methylphenyl)-1-[4-(methylsulfonyl)phenyl]-4-trifluoromethyl-1H-imidazole;

4-[2-(3-chloro-4-methylphenyl)-4-(trifluoromethyl)-1H-imidazol-1-yl]benzenesulfonamide;

2-(3-fluoro-5-methylphenyl)-1-[4-(methylsulfonyl)phenyl]-4-(trifluoromethyl)-1H-imidazole;

4-[2-(3-fluoro-5-methylphenyl)-4-(trifluoromethyl)-1H-imidazol-1-yl]benzenesulfonamide;

2-(3-methylphenyl)-1-[4-(methylsulfonyl)phenyl]-4-trifluoromethyl-1H-imidazole;

4-[2-(3-methylphenyl)-4-trifluoromethyl-1H-imidazol-1-yl]benzenesulfonamide;

1-[4-(methylsulfonyl)phenyl]-2-(3-chlorophenyl)-4-trifluoromethyl-1H-imidazole;

4-[2-(3-chlorophenyl)-4-trifluoromethyl-1H-imidazol-1-yl]benzenesulfonamide;

4-[2-phenyl-4-trifluoromethyl-1H-imidazol-1-yl]benzenesulfonamide;

4-[2-(4-methoxy-3-chlorophenyl)-4-trifluoromethyl-1H-imidazol-1-yl]benzenesulfonamide;

1-allyl-4-(4-fluorophenyl)-3-[4-(methylsulfonyl)phenyl]-5-(trifluoromethyl)-1H-pyrazole;

4-[1-ethyl-4-(4-fluorophenyl)-5-(trifluoromethyl)-1H-pyrazol-3-yl]benzenesulfonamide;

N-phenyl-[4-(4-fluorophenyl)-3-[4-(methylsulfonyl)phenyl]-5-(trifluoromethyl)-1H-pyrazol-1-yl]acetamide; ethyl [4-(4-fluorophenyl)-3-[4-(methylsulfonyl)phenyl]-5-(trifluoromethyl)-1H-pyrazol-1-yl]acetate;

4-(4-fluorophenyl)-3-[4-(methylsulfonyl)phenyl]-1-(2-phenylethyl)-1H-pyrazole;

4-(4-fluorophenyl)-3-[4-(methylsulfonyl)phenyl]-1-(2-phenylethyl)-5-(trifluoromethyl)pyrazole;

1-ethyl-4-(4-fluorophenyl)-3-[4-(methylsulfonyl)phenyl]-5-(trifluoromethyl)-1H-pyrazole;

5-(4-fluorophenyl)-4-(4-methylsulfonylphenyl)-2-trifluoromethyl-1H-imidazole;

4-[4-(methylsulfonyl)phenyl]-5-(2-thiophenyl)-2-(trifluoromethyl)-1H-imidazole;

5-(4-fluorophenyl)-2-methoxy-4-[4-(methylsulfonyl)phenyl]-6-(trifluoromethyl)pyridine;

2-ethoxy-5-(4-fluorophenyl)-4-[4-(methylsulfonyl)phenyl]-6-(trifluoromethyl)pyridine;

5-(4-fluorophenyl)-4-[4-(methylsulfonyl)phenyl]-2-(2-propynyloxy)-6-(trifluoromethyl)pyridine;

2-bromo-5-(4-fluorophenyl)-4-[4-(methylsulfonyl)phenyl]-6-(trifluoromethyl)pyridine;

4-[2-(3-chloro-4-methoxyphenyl)-4,5-difluorophenyl]benzenesulfonamide;

1-(4-fluorophenyl)-2-[4-(methylsulfonyl)phenyl]benzene;

5-difluoromethyl-4-(4-methylsulfonylphenyl)-3-phenylisoxazole;

4-[3-ethyl-5-phenylisoxazol-4-yl]benzenesulfonamide;

4-[5-difluoromethyl-3-phenylisoxazol-4-yl]benzenesulfonamide;

4-[5-hydroxymethyl-3-phenylisoxazol-4-yl]benzenesulfonamide;

4-[5-methyl-3-phenyl-isoxazol-4-yl]benzenesulfonamide;

1-[2-(4-fluorophenyl)cyclopenten-1-yl]-4-(methylsulfonyl)benzene;

1-[2-(4-fluoro-2-methylphenyl)cyclopenten-1-yl]-4-(methylsulfonyl)benzene;

1-[2-(4-chlorophenyl)cyclopenten-1-yl]-4-(methylsulfonyl)benzene;

1-[2-(2,4-dichlorophenyl)cyclopenten-1-yl]-4-(methyl sulfonyl)benzene;

1-[2-(4-trifluoromethylphenyl)cyclopenten-1-yl]-4-(methylsulfonyl)benzene;

1-[2-(4-methylthiophenyl)cyclopenten-1-yl]-4-(methylsulfonyl)benzene;

1-[2-(4-fluorophenyl)-4,4-dimethylcyclopenten-1-yl]-4-(methylsulfonyl)benzene;

4-[2-(4-fluorophenyl)-4,4-dimethylcyclopenten-1-yl]benzenesulfonamide;

1-[2-(4-chlorophenyl)-4,4-dimethylcyclopenten-1-yl]-4-(methylsulfonyl)benzene;

4-[2-(4-chlorophenyl)-4,4-dimethylcyclopenten-1-yl]benzenesulfonamide;

4-[2-(4-fluorophenyl)cyclopenten-1-yl]benzenesulfonamide;

4-[2-(4-chlorophenyl)cyclopenten-1-yl]benzenesulfonamide;

1-[2-(4-methoxyphenyl)cyclopenten-1-yl]-4-(methylsulfonyl)benzene;

1-[2-(2,3-difluorophenyl)cyclopenten-1-yl]-4-(methylsulfonyl)benzene;

4-[2-(3-fluoro-4-methoxyphenyl)cyclopenten-1-yl]benzenesulfonamide;

1-[2-(3-chloro-4-methoxyphenyl)cyclopenten-1-yl]-4-(methylsulfonyl)benzene;

4-[2-(3-chloro-4-fluorophenyl)cyclopenten-1-yl]benzenesulfonamide;

4-[2-(2-methylpyridin-5-yl)cyclopenten-1-yl]benzenesulfonamide;

ethyl 2-[4-(4-fluorophenyl)-5-[4-(methylsulfonyl) phenyl]oxazol-2-yl]-2-benzyl-acetate;

2-[4-(4-fluorophenyl)-5-[4-(methylsulfonyl)phenyl]oxazol-2-yl]acetic acid;

2-(tert-butyl)-4-(4-fluorophenyl)-5-[4-(methylsulfonyl)phenyl]oxazole;

4-(4-fluorophenyl)-5-[4-(methylsulfonyl)phenyl]-2-phenyloxazole;

4-(4-fluorophenyl)-2-methyl-5-[4-(methylsulfonyl)phenyl]oxazole;

4-[5-(3-fluoro-4-methoxyphenyl)-2-trifluoromethyl-4-oxazolyl]benzenesulfonamide;

6-chloro-7-(1,1-dimethylethyl)-2-trifluoromethyl-2H-1-benzopyran-3-carboxylic acid;

6-chloro-8-methyl-2-trifluoromethyl-2H-1-benzopyran-3-carboxylic acid;

5,5-dimethyl-3-(3-fluorophenyl)-4-methylsulfonyl-2(5H)-furanone;

6-chloro-2-trifluoromethyl-2H-1-benzothiopyran-3-carboxylic acid;

4-[5-(4-chlorophenyl)-3-(trifluoromethyl)-1H-pyrazol-1-yl]benzenesulfonamide;

4-[5-(4-methylphenyl)-3-(trifluoromethyl)-1H-pyrazol-1-yl]benzenesulfonamide;

4-[5-(3-fluoro-4-methoxyphenyl)-3-(difluoromethyl)-1H-pyrazol-1-yl]benzenesulfonamide;

3-[1-[4-(methylsulfonyl)phenyl]-4-trifluoromethyl-1H-imidazol-2-yl]pyridine;

2-methyl-5-[1-[4-(methylsulfonyl)phenyl]-4-trifluoromethyl-1H-imidazol-2-yl]pyridine;

4-[2-(5-methylpyridin-3-yl)-4-(trifluoromethyl)-1H-imidazol-1-yl]benzenesulfonamide;

4-[5-methyl-3-phenylisoxazol-4-yl]benzenesulfonamide;

4-[5-hydroxymethyl-3-phenylisoxazol-4-yl]benzenesulfonamide;

[2-trifluoromethyl-5-(3,4-difluorophenyl)-4-oxazolyl]benzenesulfonamide;

4-[2-methyl-4-phenyl-5-oxazolyl]benzenesulfonamide;

4-[5-(2-fluoro-4-methoxyphenyl)-2-trifluoromethyl-4-oxazolyl]benzenesulfonamide;

[2-(2-chloro-6-fluoro-phenylamino)-5-methyl-phenyl]-acetic acid;

N-(4-Nitro-2-phenoxy-phenyl)-methanesulfonamide or nimesulide;

N-[6-(2,4-difluoro-phenoxy)-1-oxo-indan-5-yl]-methanesulfonamide or flosulide;

N-[6-(2,4-Difluoro-phenylsulfanyl)-1-oxo-1H-inden-5-yl]-methanesulfonamide, soldium salt;

N-[5-(4-fluoro-phenylsulfanyl)-thiophen-2-yl]-methanesulfonamide;

3-(3,4-Difluoro-phenoxy)-4-(4-methanesulfonyl-phenyl)-5-methyl-5-(2,2,2-trifluoro-ethyl)-5H-furan-2-one;

(5Z)-2-amino-5-[[3,5-bis(1,1-dimethylethyl)-4-hydroxyphenyl]methylene]-4(5H)-thiazolone or darbufelone;

N-[3-(formylamino)-4-oxo-6-phenoxy-4H-1-benzopyran-7-yl]-methanesulfonamide;

(6aR,10aR)-3-(1,1-dimethylheptyl)-6a,7,10,10a-tetrahydro-1-hydroxy-6,6-dimethyl-6H-dibenzo[b,d]pyran-9-carboxylic acid;

4-[[3,5-bis(1,1-dimethylethyl)-4-hydroxyphenyl]methylene]dihydro-2-methyl-2H-1,2-oxazin-3(4H)-one;

6-dioxo-9H-purin-8-yl-cinnamic acid (B-231);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid; or

an isomer, a pharmaceutically acceptable salt, ester or prodrug thereof.

As stated above, the COX-2 inhibitors may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

Dosages and Pharmaceutical Compositions for PV Combination Therapy

By the term “effective amount” of a compound as provided herein is meant a nontoxic but sufficient amount of one or more antiviral agents in combination with one or more COX-2 inhibitor compounds to provide the desired effect. The desired effect may be to prevent, give relief from, or ameliorate PV.

As pointed out below, the exact amount of the antiviral agent and COX-2 inhibitor compound required to treat PV will vary from subject to subject, depending on the species, age, and general condition of the subject, the severity of the disease that is being treated, the particular compound(s) used, the mode of administration, such as the route and frequency of administration, and the particular compound(s) employed, and the like. Thus, it is not possible to specify an exact “effective amount.” However, an appropriate effective amount may be determined by one of ordinary skill in the art using only routine experimentation.

The pharmaceutical compositions may contain the antiviral agent and COX-2 inhibitor compound, each in the range of about 0.001 to 100 mg/kg/day for an adult, preferably in the range of about 0.1 to 50 mg/kg/day for an adult. A total daily dose of about 1 to 1000 mg of each active ingredient may be appropriate for an adult. The desired dosage may conveniently be presented in a single dose or as divided into multiple doses administered at appropriate intervals, for example, as two, three, four or more sub-doses per day. The sub-dose itself may be further divided, e.g., into a number of discrete loosely spaced administrations.

Initial treatment of a patient suffering from PV can begin with a dosage regimen as indicated above. Treatment is generally continued as necessary over a period of several weeks to several months or years until the condition or disorder has been controlled or eliminated. Patients undergoing treatment with a composition of the invention can be routinely monitored by any of the methods well known in the art to determine the effectiveness of therapy. Continuous analysis of data from such monitoring permits modification of the treatment regimen during therapy so that optimally effective amounts of drug are administered at any point in time, and so that the duration of treatment can be determined. In this way, the treatment regimen and dosing schedule can be rationally modified over the course of therapy so that the lowest amount of the COX-2 inhibitor exhibiting satisfactory effectiveness is administered, and so that administration is continued only for so long as is necessary to successfully treat the condition or disorder.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

In a combination therapy, the antiviral agent compound(s) and COX-2 inhibitor compound(s) can be administered simultaneously or at separate intervals. When administered simultaneously the anti-viral agent compound(s) and COX-2 inhibitor compound(s) can be incorporated into a single pharmaceutical composition or into separate compositions, e.g., antiviral agent compound(s) in one composition and the COX-2 inhibitor compound(s) in another composition. For instance the combination therapy, the antiviral agent compound(s) may be administered concurrently or concomitantly with the COX-2 inhibitor compound(s). The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

When separately administered, therapeutically effective amounts of antiviral agent compound(s) and COX-2 inhibitor compound(s) are administered on a different schedule. One may be administered before the other as long as the time between the two administrations falls within a therapeutically effective interval. A therapeutically effective interval is a period of time beginning when one of either (a) the antiviral agent compound(s), or (b) the COX-2 inhibitor compound(s) is administered to a mammal and ending at the limit of the beneficial effect in the treatment of PV of the combination of (a) and (b). The methods of administration of the antiviral agent compound(s) and the COX-2 inhibitor compound(s) may vary. Thus, one agent may be administered orally, while the other is administered by injection.

A specific active agent may have more than one recommended dosage range, particularly for different routes of administration. Generally, an effective amount of dosage of antiviral agent compound(s), either administered individually or in combination with other and COX-2 inhibitor compound(s), will be in the range of about 5 to about 1000 mg/kg of body weight/day, more preferably about 10 to about 750 mg/kg of body weight/day, and most conveniently from 50 to 500 mg per unit dosage form. Generally, an effective amount of dosage of COX-2 inhibitor compound(s), either administered individually or in combination with other and antiviral agent compound(s), will be in the range of about 5 to about 1000 mg/kg of body weight/day, more preferably about 10 to about 750 mg/kg of body weight/day, and most conveniently from 50 to 500 mg per unit dosage form. It is to be understood that the dosages of active component(s) may vary depending upon the requirements of each subject being treated and the severity of the viral infection.

In addition to the antiviral and COX-2 inhibitor compound(s), the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

In some embodiments, the pharmaceutical composition can include one or more antiviral agents, one or more COX-2 inhibitors, and one or more cyclooxygenase-inhibiting non-steroidal anti-inflammatory drugs (NSAID). Examples of cyclooxygenase-inhibiting NSAIDs include the well-known compounds aspirin, indomethacin, sulindac, etodolac, mefenamic acid, tolmetin, ketorolac, diclofenac, ibuprofen, naproxen, fenoprofen, ketoprofen, oxaprozin, flurbiprofen, nitroflurbiprofen, piroxicam, tenoxicam, phenylbutazone, apazone, or nimesulide or a pharmaceutically acceptable salt or derivative or prodrug thereof. In a preferred embodiment of the invention the NSAID is selected from the group comprising indomethacin, ibuprofen, naproxen, flurbiprofen or nitroflurbiprofen. In a still more preferred embodiment of the invention the NSAID is nitroflurbiprofen. In a combination therapy, the antiviral agent(s), COX-2 inhibitor compound(s), and the NSAID(s) can be administered simultaneously or at separate intervals. When administered simultaneously the the antiviral agent(s), COX-2 inhibitor compound(s), and the NSAID(s) can be incorporated into a single pharmaceutical composition or into separate compositions, e.g., the NSAID in one composition, the COX-2 inhibitor compound(s) in another composition, and the antiviral agent in, yet another composition. For instance the combination therapy, NSAID may be administered concurrently or concomitantly with the antiviral agent(s) and the COX-2 inhibitor compound(s). The term “concurrently” means the subject being treated takes one drug within about 5 minutes of taking the other drugs. The term “concomitantly” means the subject being treated takes one drug within the same treatment period of taking the other drugs. The same treatment period is preferably within twelve hours and up to forty-eight hours.

When separately administered, therapeutically effective amounts the antiviral agent(s), the COX-2 inhibitor compound(s), and the NSAID(s) are administered on a different schedule. One may be administered before the others as long as the time between the administrations falls within a therapeutically effective interval. A therapeutically effective interval is a period of time beginning when one of either (a) NSAID, or (b) the antiviral agent(s) and the COX-2 inhibitor compound(s) are administered to a mammal and ending at the limit of the beneficial effect in the treatment of PV of the combination of (a) and (b). The methods of administration of NSAID, the antiviral agent(s), and the COX-2 inhibitor compound(s) may vary. Thus, one agent may be administered orally, while the other is administered by injection.

A specific active agent may have more than one recommended dosage range, particularly for different routes of administration. Generally, an effective amount of dosage of each of the antiviral agent(s) and COX-2 inhibitors, either administered individually or in combination with NSAID, will be in the range of about 5 to about 1000 mg/kg of body weight/day, more preferably about 10 to about 750 mg/kg of body weight/day, and most conveniently from 50 to 500 mg per unit dosage form. It is to be understood that the dosages of active component(s) may vary depending upon the requirements of each subject being treated and the severity of the viral infection.

For internal infections, the pharmaceutical composition including one or more antiviral agent and one or more COX-2 inhibitor can be administered orally or parenterally at dose levels, calculated as the free base, of each of the antiviral agent and COX-2 inhibitor at 0.1 to 300 mg/kg, preferably 1.0 to 30 mg/kg of mammal body weight, and can be used in a human in a unit dosage form, administered one to four times daily in the amount of 1 to 1000 mg per unit dose.

Generally, the concentration of each of the antiviral agents and the COX-2 inhibitors in a liquid composition, such as a lotion, will be from about 0.1 wt. % to about 20 wt. %, preferably from about 0.5 wt. % to about 10 wt. %. The solution may contain other ingredients, such as emulsifiers, antioxidants or buffers. The concentration in a semi-solid or solid composition, such as a gel or a powder will be about 0.1 wt. % to about 5 wt. %, preferably about 0.5 wt. % to about 2.5 wt. %. When the topically deliverable pharmaceutical composition of the present invention is utilized to effect targeted treatment of a specific internal site, each of the antiviral agent and the COX-2 inhibitor is preferably contained in the composition in an amount of from 0.05-10 wt. %, more preferably 0.5-5 wt. %.

Routes of Administration

In therapeutic use for treating, or combating, viral infections in a mammal (i.e. human and animals) the pharmaceutical composition including the antiviral agent(s) and the COX-2 inhibitor(s) can be administered orally, parenterally, topically, rectally, or intranasally.

Parenteral administrations include injections to generate a systemic effect or injections directly to the afflicted area. Examples of parenteral administrations are subcutaneous, intravenous, intramuscular, intradermal, intrathecal, intraocular, intravetricular, and general infusion techniques.

Topical administrations include the treatment of infectious areas or organs readily accessibly by local application, such as, for example, eyes, ears including external and middle ear infections, vaginal, open and sutured or closed wounds and skin. It also includes transdermal delivery to generate a systemic effect.

The rectal administration includes the form of suppositories.

The intranasally administration includes nasal aerosol or inhalation applications.

Typically, the antiviral agent(s) and the COX-2 inhibitor(s) are administered orally, intravenously, or topically.

Pharmaceutical compositions including the antiviral agent(s) and the COX-2 inhibitor(s) may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the the antiviral agent(s) and the COX-2 inhibitor(s) can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mnnitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identificatin or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of pharmaceutical compositions with the antiviral agent(s) and the COX-2 inhibitor(s) dissolved in water and water-propylene glycol and water-polyethylene glycol systems, optionally containing suitable conventional coloring agents, flavoring agents, stabilizers and thickening agents.

The antiviral agent(s) and the COX-2 inhibitor(s) may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the antiviral agent(s) and the COX-2 inhibitor(s) may be formulated in aqueous solution, preferably in physiologically compatible buffers or physiological saline buffer. Suitable buffering agents include tri-sodium orthophosphate, sodium bicarbonate, sodium citrate, N-methyl-glucamine, L(+)-lysine and L(+)-arginine.

The compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the antiviral agent(s) and the COX-2 inhibitor(s). Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the antiviral agent(s) and the COX-2 inhibitor(s) may be in a powder form for constitution with a suitable vehicle, e.g., sterile, pyrogen-free water, before use.

For suppository administration, the pharmaceutical compositions may also be formulated by mixing the antiviral agent(s) and the COX-2 inhibitor(s) with a suitable non-irritating excipient which is solid at room temperature but liquid at rectal temperature and therefore will melt in the rectum to release the drug. Such materials include cocoa butter, beeswax and other glycerides. For administration by inhalation, the antiviral agent(s) and the COX-2 inhibitor(s) can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative, such as benzylalkonium chloride. Alternatively, for ophthalmic uses, the pharmaceutical compositions may be formulated in an ointment, such as petrolatum.

In addition to the formulations described previously, the antiviral agent(s) and the COX-2 inhibitor(s) may also be formulated as depot preparations. Such long acting formulations may be in the form of implants. The antiviral agent(s) and the COX-2 inhibitor(s) may be formulated for this route of administration with suitable polymers, hydrophobic materials, or as a sparing soluble derivative such as, without limitation, a sparingly soluble salt.

Additionally, the antiviral agent(s) and the COX-2 inhibitor(s) may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

In certain embodiments, the antiviral agent(s) and the COX-2 inhibitor(s) are applied topically. For topical applications, the pharmaceutical composition may be formulated in a suitable ointment containing the antiviral agent(s) and the COX-2 inhibitor(s) suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

The antiviral agent(s) and the COX-2 inhibitor(s) can be provided in the form of nanoparticles. Nanoparticles are particularly suitable for the topical administration of the antiviral agent(s) and the COX-2 inhibitor(s) which exhibit low water solubility, such as celecoxib.

Nanoparticles including or consisting essentially of the antiviral agent(s) or the COX-2 inhibitor(s) can be prepared according to any process previously applied to preparation of other drugs in nanoparticulate form. Suitable processes, without restriction, are illustratively disclosed for other drugs in patents and publications listed below and incorporated herein by reference.

U.S. Pat. No. 4,826,689 to Violanto & Fischer; U.S. Pat. Nos. 5,145,684; 5,298,262 to Na & Rajagopalan; U.S. Pat. No. 5,302,401 to Liversidge et al.; U.S. Pat. No. 5,336,507 to Na & Rajagopalan; U.S. Pat. No. 5,340,564 to Illig & Sarpotdar; U.S. Pat. No. 5,346,702 to Na & Rajagopalan; U.S. Pat. No. 5,352,459 to Hollister et al.; U.S. Pat. No. 5,354,560 to Lovrecich; U.S. Pat. Nos. 5,384,124; 5,429,824 to June; U.S. Pat. No. 5,503,723 to Ruddy et al.; U.S. Pat. No. 5,510,118 to Bosch et al.; U.S. Pat. No. 5,518,187 to Bruno et al.; U.S. Pat. No. 5,518,738 to Eickhoff et al.; U.S. Pat. No. 5,534,270 to De Castro; U.S. Pat. No. 5,536,508 to Canal et al.; U.S. Pat. No. 5,552,160 to Liversidge et al.; U.S. Pat. No. 5,560,931 to Eickhoffet al.; U.S. Pat. No. 5,560,932 to Bagchi et al.; U.S. Pat. No. 5,565,188 to Wong et al.; U.S. Pat. No. 5,569,448 to Wong et al.; U.S. Pat. No. 5,571,536 to Eickhoff et al.; U.S. Pat. No. 5,573,783 to Desieno & Stetsko; U.S. Pat. No. 5,580,579 to Ruddy et al.; U.S. Pat. No. 5,585,108 to Ruddy et al.; U.S. Pat. No. 5,587,143 to Wong; U.S. Pat. No. 5,591,456 to Franson et al.; U.S. Pat. No. 5,622,938 to Wong; U.S. Pat. No. 5,662,883 to Bagchi et al.; U.S. Pat. No. 5,665,331 to Bagchi et al.; U.S. Pat. No. 5,718,919 to Ruddy et al.; U.S. Pat. No. 5,747,001 to Wiedmann et al.; and International Patent Publication Nos. WO 93/25190, WO 96/24336, WO 97/14407, WO 98/35666, WO 99/65469, WO 00/18374, WO 00/27369, and WO 00/30615.

One of ordinary skill in the art can readily adapt the processes therein described to prepare the antiviral agent(s) and the COX-2 inhibitor(s) in nanoparticulate form. For instance, nanoparticles of COX-2 inhibitors may be prepared by a milling process, preferably a wet milling process in the presence of a surface modifying agent that inhibits aggregation and/or crystal growth of nanoparticles once created. In another embodiment of the invention, the nanoparticles of COX-2 inhibitors may be prepared by a precipitation process, preferably a process of precipitation in an aqueous medium from a solution of the drug in a non-aqueous solvent. The non-aqueous solvent can be a liquefied, e.g., supercritical, gas under pressure.

Patent and other literature relating to nanoparticulate drug compositions generally teach that smaller drug particle sizes advantageously increase the speed of onset of therapeutic effect, or other pharmacodynamic benefits, obtained upon administration. See, for example, U.S. Pat. Nos. 5,145,684, 5,298,262, 5,302,401, 5,336,507, 5,340,564, 5,662,883, and 5,665,331.

Smaller the drug particle size requires more grinding or milling time, energy and labor. Consequently, producing smaller particle sizes is more costly and less efficient. Thus, smaller nano-sized drug particles are generally significantly more expensive and labor-intensive to produce in quantity than larger nano-sized drug particles.

Surprisingly, it has been discovered that a COX-2 inhibitors having a weight average particle size of about 450 nm to about 1000 nm (referred to herein as a “sub-micron” formulation and particle size) exhibits onset time and bioavailability substantially equal to that of a comparative composition having a weight average particle size of about 200 to about 400 nm, as measured in vitro and in vivo. The sub-micron formulation requires less milling time and energy than the formulation comprising smaller nanoparticles with a weight average particle size in the 200-400 nm range.

It is further contemplated that certain advantages in addition to cost saving are obtainable with sub-micron as opposed to smaller particle sizes. For example, in situations where ultra-fine particles tend to agglomerate or fail to disperse in the body fluid, the slightly larger sub-micron particles can exhibit enhanced dispersion.

Accordingly, in a particularly preferred embodiment of the present invention, there is provided a pharmaceutical composition including a COX-2 inhibitor in a therapeutically effective amount, wherein the inhibitor is present in solid particles having a D₂₅ particle size of about 450 nm to about 1000 nm, and more preferably about 500 nm to about 900 nm, the composition providing at least a substantially similar Cₘₐₓ and/or at most a substantially similar Tₘₐₓ by comparison with an otherwise similar composition having a D₂₅ particle size of less than 400 nm, and/or providing a substantially greater Cₘₐₓ and/or a substantially shorter Tₘₐₓ by comparison with an otherwise similar composition having a D₂₅ particle size larger than 1000 nm. The pharmaceutical composition may also include a COX-2 inhibitor in a therapeutically effective amount, wherein the drug is present in solid particles, about 25% to 100% by weight of which have a particle size of about 450 nm to about 1000 nm, more preferably about 500 nm to about 900 nm. Alternatively, the pharmaceutical composition may include a COX-2 inhibitor in a therapeutically effective amount, wherein the drug is present in solid particles having a weight average particle size of about 450 nm to about 1000 nm, and more preferably about 500 nm to about 900 nm, the composition providing at least a substantially similar Cₘₐₓ and/or at most a substantially similar Tₘₐₓ by comparison with an otherwise similar composition having a weight average particle size of less than 400 nm, and/or providing a substantially greater Cₘₐₓ and/or a substantially shorter Tₘₐₓ by comparison with an otherwise similar composition having a weight average particle size larger than 1000 nm. For purposes of this description, “weight average particle size” can be considered synonymous with D₅₀ particle size.

Pharmaceutical compositions of the invention can be prepared by any suitable method of pharmacy which includes the step of bringing into association the selective COX-2 inhibitory drug and a suitable vehicle. An embodiment of the present invention is a composition including a therapeutically effective amount of a COX-2 inhibitor, for example celecoxib, fully dissolved in a solvent liquid including a pharmaceutically acceptable glycol ether. In this embodiment, substantially no part of the drug is suspended in particulate form in the solvent liquid.

Glycol ethers useful in the present invention preferably conform to the formula:

R¹—O—((CH₂)ₘO)ₙ—R²

wherein R¹ and R² are independently hydrogen or C₁₋₆ alkyl, C₁₋₆ alkenyl, phenyl or benzyl groups, but no more than one of R¹ and R² is hydrogen; m is an integer of 2 to about 5; and n is an integer of 1 to about 20. It is preferred that one of R¹ and R2 is a C₁₋₄ alkyl group and the other is hydrogen or a C₁₋₄ alkyl group; more preferably at least one of R¹ and R² is a methyl or ethyl group. It is preferred that m is 2. It is preferred that n is an integer of 1 to about 4, more preferably 2.

Glycol ethers used in compositions of the present invention typically have a molecular weight of about 75 to about 1 000, preferably about 75 to about 500, and more preferably about 100 to about 300. Importantly, the glycol ethers used in compositions of the present invention must be pharmaceutically acceptable and must meet all other conditions prescribed herein. Examples of glycols and glycol ethers that may be used in compositions of the present invention include, but ar enot limited to, ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monisobutyl ether, triethylene glycol dimethyl ether, triethylene glycol monoethyl ether, triethylene glycol monobutyl ether, tetraethylene glycol dimethyl ether, and mixtures thereof. See for example Flick (1998): Industrial Solvents Handbook, 5^{th} ed., Noyes Data Corporation, Westwood, N.J.

A presently preferred glycol ether solvent is diethylene glycol monoethyl ether, sometimes referred to in the art as DGME or ethoxydiglycol. It is available for example under the trademark Transcutol™ of Gattefosse Corporation.

Pharmaceutical compositions of the present invention may optionally include one or more pharmaceutically acceptable co-solvents. Examples of co-solvents suitable for use in compositions of the present invention include, but are not limited to, any glycol ether listed above; N-methylpyrrolidone; alcohols, for example isopropyl alcohol, glycerol, glycofurol, ethanol, myristyl alcohol and n-butanol; glycols not listed above, for example propylene glycol, 1,3-butanediol and polyethylene glycol such as PEG-200, PEG-350, PEG-400, PEG-540 and PEG-600, with PEG-400 being preferred; oleic and linoleic acid triglycerides, for example soybean oil; caprylic/capric triglycerides, for example Miglyol™ 812 of Huls; caprylic/capric mono- and diglycerides, for example Capmul™ MCM of Abitec; benzyl phenylformate; diethyl phthalate; ethyl oleate; triacetin; polyoxyethylene caprylic/capric glycerides such as polyoxyethylene (8) caprylic/capric mono- and diglycerides, for example Labrasol™ of Gattefosse; medium chain triglycerides; propylene glycol fatty acid esters, for example propylene glycol laurate; oils, for example corn oil, mineral oil, cottonseed oil, peanut oil, sesame seed oil and polyoxyethylene (35) castor oil, for example Cremophor™ EL of BASF; polyoxyethylene glyceryl trioleate, for example Tagat™ TO of Goldschmidt; and lower alkyl esters of fatty acids, for example ethyl butyrate, ethyl caprylate and ethyl oleate.

The pharmaceutical composition may also include permeation enhancers. Permeation enhancers aid in the delivery of the antiviral agent(s) and the COX-2 inhibitor(s) across the skin. As suitable permeation enhancers for use with the the antiviral agent(s) and the COX-2 inhibitor(s) of the present invention, terpenes and fatty alcohols are particularly preferred. Examples thereof include, but are not limited to, ethanol, isopropanol, 1,3-butanediol, oleyl alcohol, thymol, menthol, carvone, carveol, citral, dihydrocarveol, dihydrocarvone, neumenthol, isopulegol, terpene-4-ol, menthone, pulegol, camphor, geraniol, α-terpineol, linalol, carvacrol, t-anethole, isomers thereof, racemic mixtures thereof, and mixtures thereof. Fatty acids also may be used as permeation enhancers in the present invention. Additionally, it has been discovered that parecoxib can be used as a permeation enhancer for other COX-2 inhibitors. Combinations of permeation enhancers can be used as long as they are effective in delivering the desired amount of the antiviral agent(s) and the COX-2 inhibitor(s) to the patient.

The dosage form of the pharmaceutical compositions of the present invention can be any of those typically used to topically administer a medication such as a patch, tape, cataplasm, poultice, cream, paste or ointment and can be formulated according to conventional methods known in the art. The amount of the antiviral agent(s) and the COX-2 inhibitor(s) contained in the pharmaceutical composition is based on the desired amount to be administered, the properties of the inhibitor, the properties of the permeation enhancer and the type of treatment to be effected.

A non-limiting exemplary patch that can be used in the present invention includes a) a backing layer, (b) an adhesive layer and c) at least one antiviral agent and at least one COX-2 inhibitor which may be incorporated into the adhesive layer or separated from the adhesive layer. The backing layer should preferably be thin and made of a soft and flexible material which can change its form or shape in agreement with the motion of the subject of the treatment. It includes nonwoven fabrics, woven fabrics, flannels and spandex fabrics, and laminates derived from these materials and a polyethylene film, an ethylene-vinyl acetate film, a polyurethane film or the like, as well as polyvinyl chloride films, polyethylene films, polyurethane films, aluminum deposited films and so forth, either as they are or in the form of composite films derived therefrom. The backing layer may be either perforated to allow diffusion or perspiration moisture or impermeable in order to improve the permeability of the skin by occlusion of moisture.

The function of the adhesive layer is to provide a satisfactory level of adhesiveness to the skin of the subject. This adhesiveness can be provided by certain macromolecular substances. Examples of such macromolecular substance are gelatin, agar, alginic acid, mannan, carboxymethylcellulose, methylcellulose, polyvinyl alcohol, natural rubber, polyisoprene, polybutadiene, styrene-isoprene-styrene block copolymers, polyacrylic esters, polymethacrylic esters, acrylic ester-methacrylic ester copolymers, acrylic acid-acrylic ester-vinyl acetate copolymers and petroleum resins.

These macromolecular substances may be used either singly or in combination of two or more. When a natural rubber is used as the macromolecular substance, it is recommendable to use a composition composed of 30-70% (% by weight; hereinafter the same shall apply) of the rubber component, 30-60% of a tackifier resin, not more than 20% of a softening agent and 0.01-2% of an antioxidant. When a styrene-isoprene-styrene block copolymer is used as the macromolecular substance, it is recommendable to use a composition composed of 20-50% of said copolymer, 25-60% of a tackifier resin, 5-20% of a liquid rubber and 0.01-2% of an antioxidant.

As the tackifier resin mentioned above, there may be mentioned, for example, alicyclic saturated hydrocarbon petroleum resins, rosin, rosin glycerol ester, hydrogenated rosin, hydrogenated rosin glycerol ester, hydrogenated rosen pentaerythritol ester, cumaroneindene resins, polyterpenes, terpene-phenolic resins, cycloaliphatic hydrocarbon resins, alkyl aromatic hydrocarbon resins, hydrocarbon resins, aromatic hydrocarbon resins, and phenolic resins. The antioxidant includes, but is not limited to, dibutylhydroxytoluene (BHT) and the softening agent includes, but is not limited to, liquid paraffin and petrolatum.

The above-mentioned components generally contain trace amounts of metals as impurities, which can promote decomposition of the active agent during storage and decrease the storage stability of plaster products. In accordance with the invention, a metal sequestering agent can be incorporated into the adhesive base composition, whereby metals are seized and held by said agent and accordingly promoted decomposition of the pharmacologically active component can be avoided, even during a long period of storing of the plasters. The sequestering agent to be used in accordance with the invention includes, among others, EDTA, potassium polyphosphate, sodium polyphosphate, potassium metaphosphate, sodium metaphosphate, dimethylglyoxime, 8-hydroxyquinoline, nitrilotriacetic acid, dihydroxyethylglycine, gluconic acid, citric acid and tartaric acid. These are recommendably used in an amount of 0.01-2%.

The adhesive base preparation components should be used in such relative amounts that can give satisfactory adhesive characteristics (tack, adhesive strength, cohesion strength) and satisfactory percutaneous absorption, which are fundamental to the final dosage form preparation. The allowable addition levels given above for the respective components have been established from such point of view.

The antiviral agent(s) and the COX-2 inhibitor(s) may be present in dissolved or solid form. If the active agent is in solid form, it may be advantageous to use a small particle size, e.g. micronized powder or nanoparticles as described above. Suitable solvents and/or permeation enhancers may be added in order to improve transport of the active agent. The combination constituents should desirably be selected with the control of drug release and the inhibition of skin irritation being taken into consideration. In the practice of the invention, a skin irritation reducing agent, such as vitamin E, glycyrrhetic acid or diphenhydramine, may be added. The amount of the adhesive preparation, with or without incorporated active agent, to be spread on the support is generally, but not limited to, 10-2000 g/m2.

A particular feature of the present invention is that the dosage form can be designed so that the drug penetrates the skin to deliver a pharmaceutically effective amount of the drug to a target site such as dermal, epidermal, subcutaneous and articular organs and tissues while maintaining the systemic levels of the drug no greater than the pharmaceutically effective level, preferably at systemic levels less than the pharmaceutically effective level.

In another embodiment of the present invention, the dosage form can be administered topically to deliver amounts of the antiviral agent(s) and the COX-2 inhibitor(s) sufficient to achieve systemic plasma levels at or above the therapeutically effective concentration to achieve systemic treatment with the drug.

Efficacy of Combinations of COX-2 Inhibitors and Antiviral Agents in Treating PV

The efficacy of treating PV with the combination of COX-2 inhibitors and antiviral agents can be ascertained via several models known in the art. For example a Rabbit oral papillomavirus model described by Christensen et al. in. Virology. 269(2): 451-61 (2000); a Canine oral papillomavirus model discussed by Nicholls et al. in Virology. 265: 365-374 (1999); a Bovine papillomavirus model described by McBride et al. in Proc. Natl. Acad. Sci. USA, Vol. 97, 5534-5539 (2000); Xenograft mouse models employing human tissue fragments implanted in mice discussed by Kreider et al. in Virology 177:415-417 (2000), by Bonnez et al. in Virology 197:455-458 (1993), and by Brandsma et al. in J. Virol. 69: 2716-2721 (1995); aXenograft mouse model employing human cells implanted in mice described by Sterling et al. J Virol, 64: 6305-7 (1990); Xenograft mouse models employing animal tissue fragments implanted in mice discussed by Lobe et al. in Antiviral Research, 40: 57-71 (1998), and by Pawellek et al. in Antimicrob. Agents Chemother. 45: 1014-1021. (2001); a Non-human primate papillomavirus model described by Ostrow et al. in PNAS 87: 8170-8174, and a topical Cottontail Rabbit Papillomavirus Animal Model described below.

This invention will be more fully described by way of the following Examples but is not limited to these Examples.

### EXAMPLES

As a way of measuring the skin drug permeation properties of the COX-2 inhibitors, a Franz diffusion cell was provided utilizing cadaver skin as the membrane and a 1% Tween 80 solution as the receptor phase. Frozen cadaver skin was thawed at room temperature and punched with a 20 mm puncher. The receptor compartment of the Franz diffusion cell was filled with 1% Tween 80 solution and the diffusion cells maintained at 32° C. A 6% polyethylene glycol-20-oleyl ether is also suitable as a receptor fluid. The skin was mounted on the receptor, covered with the cup and fastened by a clamp. The air bubbles were removed from the receptor fluid and it was allowed to equilibrate for 30 minutes. COX-2 pharmaceutical compositions, according to the present invention, were brought into contact with the cadaver skin and the amount of drug which permeated through the cadaver skin in a 24 hour period was determined by high performance liquid chromatography.

### Test Example 1

Pharmaceutical compositions made up of drug saturated solutions of celecoxib formulated with 70% aqueous ethanol, ethanol, polyethylene glycol having a molecular weight of 400 and propylene glycol as permeation enhancers were made and used as test compositions with the Franz diffusion cell discussed above to ascertain the drug flux through the skin. The results are shown in Table 1.

### Test Example 2

Valdecoxib pharmaceutical compositions were prepared in an identical manner as in Test Example 1 and the flux of the drug through the cadaver skin measured in the same manner. The results are also shown in Table 1.

TABLE 1

| | Drug Saturated Solution | | | | | | | |
|---|---|---|---|---|---|---|---|---|
| | Celecoxib | | | | Valdecoxib | | | |
| Formulation | | | PE | | | | PE | |
| Active | 70% | | G | | 70% | | G | |
| Vehicle | EtOH | EtOH | 400 | PG | EtOH | EtOH | 400 | PG |
| Solubility | 15.2 | 91.4 | 297 | 33.3 | 12.7 | 7.48 | 210 | 23.6 |
| (mg/ml) | | | | | | | | |
| Flux | 15.7 ± | 5.62 ± | UD | UD | 12.8 ± | 1.44 ± | UD | UD |
| (μg/cm² · day) | 3.83 | 1.49 | | | 4.96 | 0.54 | | |

### Test Example 3

A pharmaceutical composition containing parecoxib as the COX-2 inhibitor was formulated with a 70% aqueous ethanol solution and tested for its delivery of the drug across the cadaver skin in the same manner as in the previous test examples. The solubility and skin flux of the celecoxib, valdecoxib and parecoxib pharmaceutical compositions are shown for comparison purposes in Table 2.

TABLE 2

| | Solubility | Skin Flux |
|---|---|---|
| COX-2 | (mg/ml) | (μg/cm² · day) |
| Celecoxib | 15.2 | 15.7 ± 3.83 |
| Valdecoxib | 12.7 | 12.8 ± 4.96 |
| Parecoxib | 386 | 254 ± 164 |

### Test Example 4

Pharmaceutical compositions containing 5% oleyl alcohol and 3% thymol were prepared for celecoxib, valdecoxib and parecoxib. These compositions were tested for enhanced skin permeation properties. The results are shown in Table 3.

TABLE 3

| | Skin Flux | Enhancement |
|---|---|---|
| COX-2 | (μg/cm² · day) | Factor |
| Celecoxib | 21.7 ± 4.6 | 1.4 |
| Valdecoxib | 323 ± 21 | 25 |
| Parecoxib | 1210 ± 58.0 | 4.8 |

### Test Example 5

A valdecoxib pharmaceutical composition was prepared using different combinations of water, ethanol, isopropanol, 1,3-butanediol, oleyl alcohol and thymol as vehicles and skin permeation enhancers. The compositions were tested for the solubility of valdecoxib and the ability of the composition to deliver valdecoxib across the cadaver skin membrane. The results are shown in Table 4.

TABLE 4

| Ingredients | % w/w | | |
|---|---|---|---|
| Water | 30 | 33 | 30 |
| Ethanol | 62 | 62 | 30 |
| Isopropanol | | | 10 |
| 1,3-Butanediol | | | 22 |
| Oleyl Alcohol | 5 | 5 | 5 |
| Thymol | 3 | | 3 |
| Solubility | 22.0 | 18.5 | 13.4 |
| (mg/ml) | | | |
| Skin Flux | 441 ± 160 | 287 ± 23.9 | 302 ± 48.9 |
| (μg/cm² · day) | | | |

### Test Example 6

Solutions and gels of celecoxib and valdecoxib pharmaceutical compositions were prepared and tested for their skin permeation properties. The results are shown in Table 5.

TABLE 5

| | Celecoxib | | Valdecoxib | |
|---|---|---|---|---|
| Formulation | Solution* | Gel** | Solution* | Gel** |
| Concentra- | 15.2 | 10 | 12.7 | 10 |
| tion (mg/ml) | | | | |
| Amount | 250 μl | 50 mg | 250 μl | 50 mg |
| Applied | | | | |
| Occlusive | Y | N | Y | N |
| Skin Flux | 15.7 ± 3.83 | 3.82 ± 3.36 | 12.8 ± 4.96 | 11.3 ± 6.48 |
| (μg/cm² · | | | | |
| day) | | | | |
| Drug in | 3.92 ± 0.79 | 2.36 ± 1.06 | 9.27 ± 3.84 | 1.81 ± 1.87 |
| Epidermis | | | | |
| (μg) | | | | |
| Drug in | 2.50 ± 1.53 | 1.22 ± 0.51 | 0.543 ± 0.525 | UD |
| Dermis (μg) | | | | |

*Drug saturated in a 70% ethanol solution
**1% of the drug in a 70% ethanol, 3% KLUCEL gel

### Test Example 7

Celecoxib and valdecoxib pharmaceutical compositions were prepared in which 5% parecoxib was also present as a permeation enhancer. The flux of the celecoxib and parecoxib across the cadaver skin membrane was measured and the enhancement factor calculated. The results are shown in Table 6.

TABLE 6

| | Saturated Cb in | | Saturated Vb in | |
|---|---|---|---|---|
| | 5% Pb, 67% EtOH | | 5% Pb, 67% EtOH | |
| Formulation | Cb | Pb | Vb | Pb |
| Concentration | 15.9 | 49.4 | 19.2 | 49.7 |
| (mg/ml) | | | | |
| Flux | 183 ± 153 | 74.7 ± 14.7 | 108 ± 16.7 | 64.1 ± 11.3 |
| (μg/cm² · day) | | | | |
| Enhancement | 11.5 | | 8.4 | |
| Factor | | | | |

As illustrated in Tables 1-6, COX-2 inhibitors can be effectively administered to a patient by topical application. Moreover, parecoxib can unexpectedly be used as a permeation enhancer and increase the transdermal delivery of selective COX-2 drugs across the skin.

Cottontail Rabbit Model

Rabbit papillomas may be induced in domestic rabbits by inoculating viral particles or isolated viral DNA onto scarified skin sites. Since live viral particles are difficult to obtain, we used a molecularly cloned viral DNA, which is prepared and injected into rabbits as described below.

CRPV Infectious Clone. An infectious clone of Cottontail Rabbit Papillomavirus (CRPV), called CRPV-pLA2 in E. coli HB 101, was purchased from the American Type Culture Collection (ATCC), Manassas, Va. The 7.8 kb CRPV insert was cloned from the cottontail rabbit papilloma virus Washington B strain (Nasseri 1987). The CRPV genome was inserted at the Sal I site of pLA2 resulting in an 11.3 kb recombinant plasmid called CRPV-pLA2 (Nasseri 1989).

Plasmid Isolation. E. coli HB101 containing CRPV-pLA2 was reconstituted using LB Broth (Gibco-BRL) containing 100 μg/ml ampicillin. One drop of the reconstituted culture was transferred to LB agar (Gibco-BRL)+100 μg/ml ampicillin and isolation streaked. The plate was incubated overnight at 37° C. The next day, a single colony was picked from the plate and isolation streaked onto a second LB agar plate+100 μg/ml ampicillin. This procedure was repeated a third time to ensure that only those bacterial cells containing the ampicillin resistance gene located on the CRPV-pLA2 plasmid were isolated. One well-isolated colony of E. coli HB101 was then picked and transferred to 2 ml LB broth+100 μg/ml ampicillin. The culture was incubated with constant mixing at 37° C. for 6 hours. The log phase culture was then transferred to a two liter Erlenmeyer flask containing 500 ml LB broth +100 μg/ml ampicillin and shaken overnight at 150 rpm, 37° C. The next day the turbid culture was transferred to multiple 250 ml Nalgene centrifuge bottles, centrifuged at 6000×g in a Sorval GSA rotor for 15 minutes at 4° C. The supernatant was discarded and plasmid DNA was extracted from each bacterial pellet using Qiagen's EndoFree Plasmid Maxi Kit according to the manufacturer's directions. Purified CRPV-pLA2 DNA was resuspended in endotoxin-free TE buffer, pH 8.0. Plasmid concentration was determined by UV spectrophotometry and purity by analysis on an agarose gel.

Gene Gun procedure. Supercoiled plasmids were purified and precipitated onto gold particles (average diameter 1.6 um), at a ratio of lug DNA:0.5 mg gold, in 0.1 M spermidine and 2.5 M CaCl₂ during a 10 min incubation at 20° C. The DNA-coated gold particles were pelleted at 12 000 rpm for 30 s, washed three times with 100% ethanol, and resuspended at 2 ug DNA/mg gold/ml ethanol. The DNA-gold-ethanol suspension was introduced into a 22″ section of Tefzel tubing (1/8″ outside diameter, 3/32″ internal diameter) (McMaster-Carr, Elmhurst, Ill.). Particles were allowed to settle onto the bottom of the tubing and the ethanol was then evacuated using a peristaltic pump. The tubing was then rotated at 20 rpm for 30 s in a device (BioRad, Inc.) designed to distribute the gold evenly over the inner walls of the tubing. Rotation was continued as the DNA-gold was dried under a continuous stream of nitrogen gas delivered at 250 ml/min. The tubing was sliced into ½″ lengths to generate ‘shots’ containing 1 ug DNA/0.5 mg gold. The shots were loaded into a 12-chamber barrel of a helium-driven Helios Gene Delivery Device (BioRad, Inc.)

Rabbit Model. Female New Zealand White (NZW) rabbits, each weighing 2-3 kg were used. Water and high fiber rabbit chow were provided ad libitum. For viral DNA inoculation, rabbits were anesthetized by administering a mixture of ketamine hydrochloride (Ketaset®, 100 mg/ml) and xylazine (Anased®, 20 mg/ml). Rabbits were shaved on each flank and residual hair removed by the use of Nare™, a depilatory agent. The CRPV-pLA2 clone on carrier gold particles were injected into the epidermis of anesthetized rabbits using the Helios Gene Gun at 400 psi pressure. The inoculated skin sites developed varying degree of redness along with some brown coloration due to the presence of the gold particles both within and on the skin. We inoculated three sites on each flank for a total of six sites per rabbit. Inoculated sites were inspected weekly for 8-16 weeks, depending on the experimental designs. For ruhe number and size of papillomas at individual inoculation sites were recorded.

Immediately after injection, the target sites are recognizable by redness and an outer area of faint traces of gold on the surface of the skin. Six skin sites per rabbit were injected and inoculated sites showed small pink nodules (˜10 nodules per site) of about one mm in diameter as early as 16-18 days post inoculation. There was no difference in the rates of papilloma formation between sites inoculated at 350 and 400 p.s.i. In experiment one, 24 of 24 sites (100%) inoculated in four of four rabbits formed papillomas, with an average of ten per site (240 papillomas/24 sites) at four weeks after inoculation. Similar findings were observed in the second studies. At four weeks post inoculation, the total lesion areas were about 10-100 mm² and these increased to 50-500 mm² by eight weeks and ˜5000 mm² by 16 weeks post inoculation. In both of these study groups, we observed marked variability in the size of warts produced among different animals and among the six sites from the same animal. We noted that warts appeared earlier and grew at a faster rate in some animals compared to others. Since we used out-bred rabbits, this variation in response is likely due to the host immune status that is known to affect wart development in clinical settings. Papillomas were recognizable grossly in most animals by four weeks after inoculation. A few additional lesions are observable in some inoculated sites for up to about 7 weeks.

Histologic evaluation of lesions collected at euthanasia revealed the typical features of viral papillomas, including hyperplasia, acanthosis, parakeratosis and koilocytosis (data not shown). The presence of CRPV DNA was confirmed by in situ hybridization staining of formalin-fixed tissue samples. DNAs were extracted from papillomas and amplified by polymerase chain reaction (PCR) using CRPV primers CR986C (5′-GCT ATC CTG TGC GCA GGG C-3′) and CR1440N (5′-GGT TGT CAC AGT CTA AAC AGT CC-3′) that flank a 455 bp region of the CRPV E7-E1 genes. Using this PCR assay, CRPV DNAs were detected in papilloma samples collected from all stages of papilloma growth (data not shown).

COX-2 EXPRESSION. COX-2 protein plays an important role in inflammation and in cell proliferation as a result of the stimulation of prostaglandin E2 synthesis. A key feature of papillomavirus infection is the viral induced hyperplasia which is related to the ability of the virus to interfere with the regulation of normal cell cycle. The growth promoting property of COX-2 may be involved in the pathogenetic mechanism of viral induced abnormal cell growth and development. In addition to the effect of host response to infection, certain papillomavirus proteins may also contribute to the over-expression of COX-2 in wart tissue. For example, certain viral proteins may indirectly lead to over expression of COX-2. For papillomaviruses, two proteins, E6 and E7, are known to alter host cell maturation and growth, leading to the formation of epithelial hyperplasia and papillomas. One of the effects of PV E6 is the binding and subsequent degradation of the tumor suppressor protein p53 via the ubiquitin proteolysis pathway. Among its varied functions, p53 is known to suppress COX-2 gene expression. Thus, by negating or otherwise reducing the function of p53, the E6 protein might indirectly induce COX-2 expression in the infected tissues. Although less well defined, the E7 protein may also lead to COX-2 expression by the activation of the AP-1 family of transcription factors resulting in the activation of COX-2 transcription.

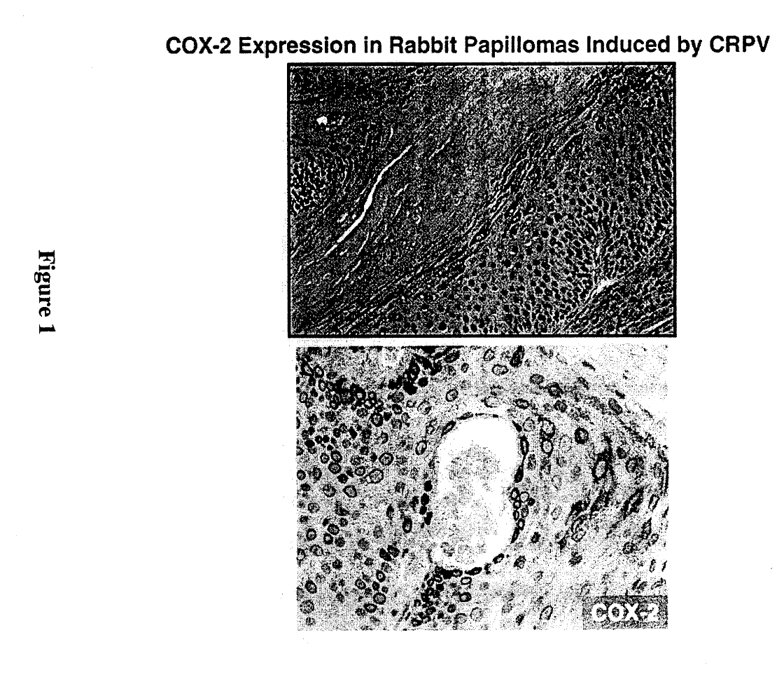

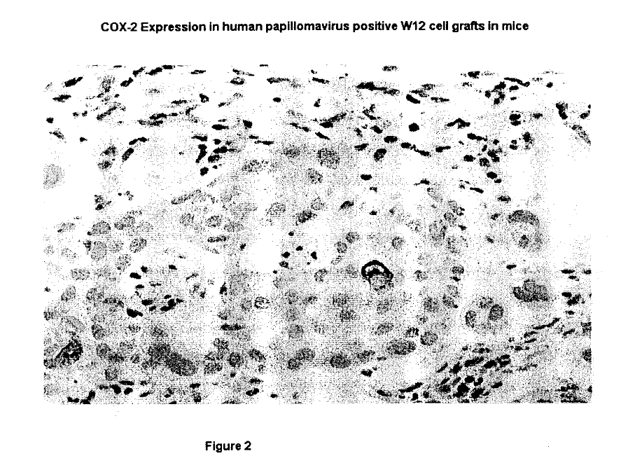

Despite these observations, there has been no report on the expression of COX-2 in papillomavirus infected cells or tissues. Studies were performed to investigate COX-2 expression in papilloma tissues collected from CRPV infected rabbits. Formalin-fixed sections were stained for COX-2 protein using a goat anti-rat COX-2 antibody followed by streptavidin-HRP and DAB substrate detection procedure (DAKO). FIG. 1 shows that COX-2 immunoreactivity was localized predominantly to cells within the granular and the spinous layers. Importantly, these layers of the epidermis are known to be the sites of viral DNA amplification. However, there was also evidence of the presence of COX-2 in the basal layer and vascular endothelial cells. The intracellular distribution of COX-2 immunoreactivity is perinuclear and cytoplasmic in all labeled cells. COX-2 protein was detected in wart samples from various stages of growth, suggesting that COX-2 was expressed in early (3-4 weeks) as well as later stages (24 weeks) of wart growth. This observation implies that COX-2 overexpression is an early and continuous event in wart pathogenesis. In addition to demonstrating the presence of COX-2 protein in rabbit warts, FIG. 2 shows the presence of COX-2 in human papillomavirus infected cells and cell grafts obtained from mouse models. COX-2 may promote epithelial hyperplasia and wart formation in several ways, including the stimulation of cell growth, inhibition of immune cells, inhibition of apoptosis, and promotion of angiogenesis.

Treatment Regimen. Test animals are divided into separate groups consisting of non-treated control, vehicle or placebo control and drug treated groups. The vehicle control consists of the inert components of the topical formulations, but without the drugs, i.e., the composition containing the COX-2 inhibitor and the anti-viral agent. Animals are treated with the topical formulations of the drug preparations once a day for a period of four weeks, with therapy beginning at various times after inoculation. A measured amount of the topical formulation is applied liberally to each inoculation site. After treatment, collars are put on the animals for 1-2 hours to prevent licking of the target skin sites. After the termination of therapy, animals can be kept for an additional period of 2-4 weeks depending on experimental design.

Evaluation of Drug Efficacy. The growth of the papillomas can be measured at weekly intervals by using a digital caliper. Measurements can be taken as length, width and height. Papilloma volume can be calculated by multiplying the height, width and length of each wart and expressed in mm³. For each animal, wart size on each flank can be added together to produce a single value of total wart volume. Drug efficacy of the combination therapy can be determined for an individual animal by comparing the wart volume of treated animals versus vehicle or placebo animals. In animals that have shown total regression of warts, drug efficacy can also be recorded as percent skin sites with warts of treated animals versus vehicle or placebo animals. In addition, the specific COX-2 inhibitors and antiviral agents, and amounts of each component in the pharmaceutical composition can be determined by comparing the recorded efficacy of multiple compositions, each having different active ingredients and/or amount of active ingredients.

Without further elaboration, it is believed that one skilled in the art can, using the preceding description, practice the present invention to its fullest extent. The foregoing detailed description is given for clearness of understanding only, and no unnecessary limitations should be understood therefrom, as modifications within the scope of the invention may become apparent to those skilled in the art.

What is claimed is:

1. A method of treating PV comprising administering to a mammal, in need of treatment for PV, a therapeutically effective amount of a COX-2 inhibitor or a pharmaceutically acceptable salt thereof and a therapeutically effective amount of an antiviral agent or a pharmaceutically acceptable salt thereof.

2. The method of claim 1, wherein the effective amount of the COX-2 inhibitor and antiviral agent is administered to the mammal topically.

3. The method of claim 1, wherein the COX-2 inhibitor and antiviral agent are included as components of a pharmaceutical composition in which the pharmaceutical composition further comprises a permeation enhancer.

4. The method of claims 1, wherein the COX-2 inhibitor is a compound having the structure of Formula III 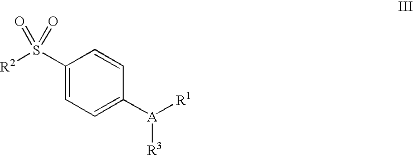
wherein A is a substituent selected from partially unsaturated or unsaturated heterocyclyl and partially unsaturated or unsaturated carbocyclic rings;
wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is methyl or amino; and
wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

5. The method of claim 4, wherein the COX-2 inhibitor compound is celecoxib (A-21), valdecoxib (A-22), deracoxib (A-23), rofecoxib (A-24), etoricoxib (A-25), JTE-522 (A-26), or parecoxib (A-27).

6. The method of claim 5, wherein the COX-2 inhibitor is at least one member selected from the group consisting of celecoxib, valdecoxib and parecoxib.

7. The method of claim 1, wherein the COX-2 inhibitor is a compound selected from the group consisting of

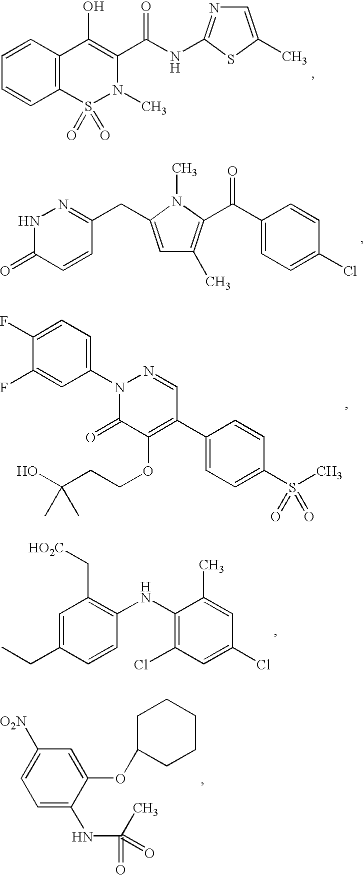

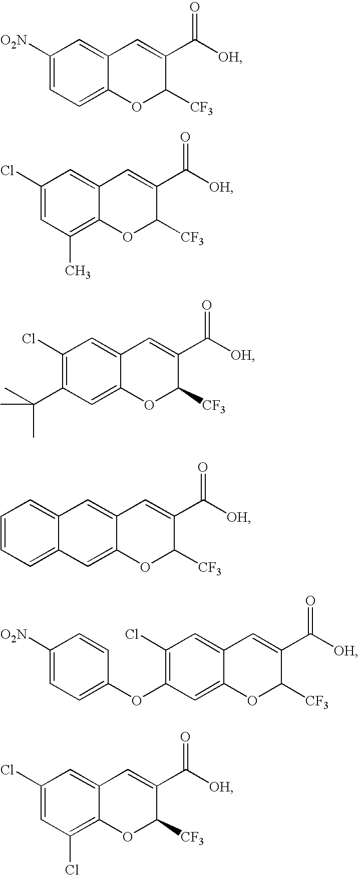

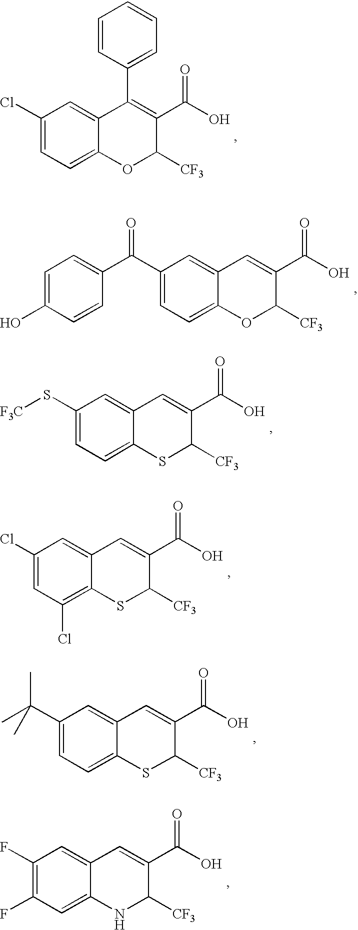

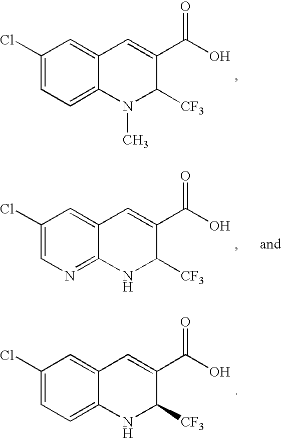

8. The method of claim 1, wherein the COX-2 inhibitor is selected from 6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphtho[2,1-b]pyran-3-carboxylic acid;
7-(1, 1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-, carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(11,1-dimethylethyl)-2-pentafluoroethyl-2H-1-benzopyran-3-carboxylic acid;
6-chloro-2-trifluoromethyl-2H-1-benzothiopyran-3-carboxylic acid;
3-[(3-Chloro-phenyl)-(4-methanesulfonyl-phenyl)-methylene]-dhydro-furan-2-one;
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl] benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl] spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl] spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-fluorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-metboxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-7-(1,1-dimethylethyl)-2-trifluoromethyl-2H-1-benzopyran-3-carboxylic acid;
6-chloro-8-methyl-2-trifluoromethyl-2H-1-benzopyran-3-carboxylic acid;
5,5-dimethyl-3-(3-fluorophenyl)-4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenyl isoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide;
4-[5-(2-fluoro-4-methoxyphenyl)-2-trifluoromethyl-4-oxazolyl]benzenesulfonamide;
[2-(2-chloro-6-fluoro-phenylamino)-5-methyl-phenyl]-acetic acid;
N-(4-Nitro-2-phenoxy-phenyl)-methanesulfonamide or nimesulide;
N-[6-(2,4-difluoro-phenoxy)-1-oxo-indan-5-yl]-methanesulfonamide or flosulide;
N-[6-(2,4-Difluoro-phenylsulfanyl)-1-oxo-1H-inden-5-yl]-methanesulfonamide, soldium salt;
N-[5-(4-fluoro-phenylsulfanyl)-thiophen-2-yl]-methanesulfonamide;
3-(3,4-Difluoro-phenoxy)-4-(4-methanesulfonyl-phenyl)-5-methyl-5-(2,2,2-trifluoro-ethyl)-5H-furan-2-one;
(5Z)-2-amino-5-[[3,5-bis(1,1-dimethylethyl)-4-hydroxyphenyl]methylene]-4(5H)-thiazolone or darbufelone;
N-[3-(formylamino)-4-oxo-6-phenoxy-4H-1-benzopyran-7-yl]-methanesulfonamide;
(6aR,10aR)-3-(1,1-dimethylheptyl)-6a,7,10,10a-tetrahydro-1-hydroxy-6,6-dimethyl-6H-dibenzo[b,d]pyran-9-carboxylic acid;
4-[[3,5-bis(1,1-dimethylethyl)-4-hydroxyphenyl]methylene]dihydro-2-methyl-2H-1,2-oxazin-3(4H)-one;
6-dioxo-9H-purin-8-yl-cinnamic acid (B-231);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methyl sulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;
[2-(2,4-dichloro-6-ethyl-3,5-dimethyl-phenylamino)-5-propyl-phenyl]-acetic acid; or
an isomer, a pharmaceutically acceptable salt, ester or prodrug thereof.

9. The method of claim 1, wherein the COX-2 inhibitor has the formula 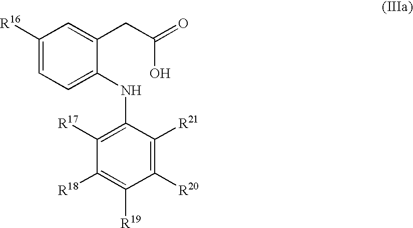
or an isomer, a pharmaceutically acceptable salt, ester, or prodrug thereof;
wherein
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R⁶ is ethyl and R¹⁹ is H.

10. The method of claim 1, wherein the COX-2 inhibitor has the formula 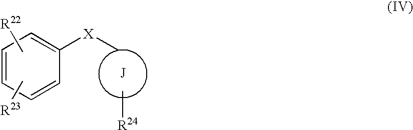
or an isomer, a pharmaceutically acceptable salt, an ester, or a prodrug thereof,
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

11. The method of claim 1, wherein the COX-2 inhibitor has the formula 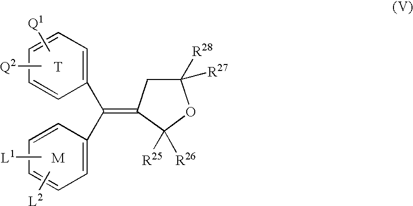
or an isomer, a pharmaceutically acceptable salt, an ester, or a prodrug thereof,
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

12. The method of claim 3, wherein the permeation enhancer comprises a compound selected from the group consisting of ethanol, isopropanol, 1,3-butanediol, oleyl alcohol, thymol, menthol, carvone, carveol, citral, dihydrocarveol, dihydrocarvone, neumenthol, isopulegol, terpene-4-ol, menthone, pulegol, camphor, geraniol, α-terpineol, linalol, carvacrol, t-anethole, and parecoxib.

13. The method of claim 12, wherein the permeation enhancer comprises a compound selected from the group of ethanol, isopropanol, 1,3-butanediol, oleyl alcohol, thymol, and paracoxib.

14. The method of claim 13, wherein the permeation enhancer comprises paracoxib.

15. The method of claim 4, wherein the permeation enhancer comprises a compound selected from the group consisting of ethanol, isopropanol, 1,3-butanediol, oleyl alcohol, thymol, menthol, carvone, carveol, citral, dihydrocarveol, dihydrocarvone, neumenthol, isopulegol, terpene-4-ol, menthone, pulegol, camphor, geraniol, α-terpineol, linalol, carvacrol, t-anethole, and parecoxib.

16. The method of claim 15, wherein the permeation enhancer comprises a compound selected from the group of ethanol, isopropanol, 1,3-butanediol, oleyl alcohol, thymol, and paracoxib.

17. The method of claim 16, wherein the permeation enhancer comprises paracoxib.

18. The method of claim 1, wherein the selective COX-2 inhibitor is contained in the pharmaceutical composition in an amount of from 0.05-10 wt. %.

19. The method of claim 1, wherein the COX-2 inhibitor is a component in a pharmaceutical composition which further comprises a glycol ether of the formula
R¹—O—((CH₂)ₘO)ₙ—R²
wherein R¹ and R² are independently hydrogen or C₁₋₆ alkyl, C₁₋₆ alkenyl, phenyl or benzyl group, with only one of R¹ and R² being hydrogen; m is an integer of 2 to 5 and n is an integer of 1 to 20.

20. The method of claim 1, wherein at least 25% by weight of the COX-2 inhibitor is in the form of nanoparticles having a particle size from about 450 to about 900 nm.

21. The method of claim 15, wherein at least 50% by weight of the COX-2 inhibitor is in the form of nanoparticles having a particle size from about 450 to about 900 nm.

22. The method of claim 16, wherein at least 75% by weight of the COX-2 inhibitor is in the form of nanoparticles having a particle size from about 450 to about 900 nm.

23. A method of treating PV, comprising topically applying a pharmaceutical composition comprising a COX-2 inhibitor and an antiviral agent, both in a concentration sufficient to obtain the therapeutically effective amount of the COX-2 inhibitor and antiviral agent in tissue infected with PV.

24. The method of claim 23, wherein the COX-2 inhibitor is a compound having the structure of Formula III 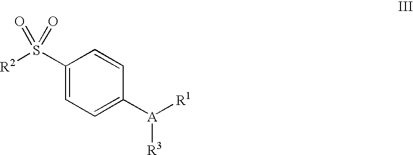
wherein A is a substituent selected from partially unsaturated or unsaturated heterocyclyl and partially unsaturated or unsaturated carbocyclic rings;
wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is methyl or amino; and
wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

25. The method of claim 24, wherein the COX-2 inhibitor compound is celecoxib (A-21), valdecoxib (A-22), deracoxib (A-23), rofecoxib (A-24), etoricoxib (A-25), JTE-522 (A-26), or parecoxib (A-27).

26. The method of claim 25, wherein the COX-2 inhibitor is at least one member selected from the group consisting of celecoxib, valdecoxib and parecoxib.

27. The method of claim 23, wherein the COX-2 inhibitor is a compound selected from the group consisting of

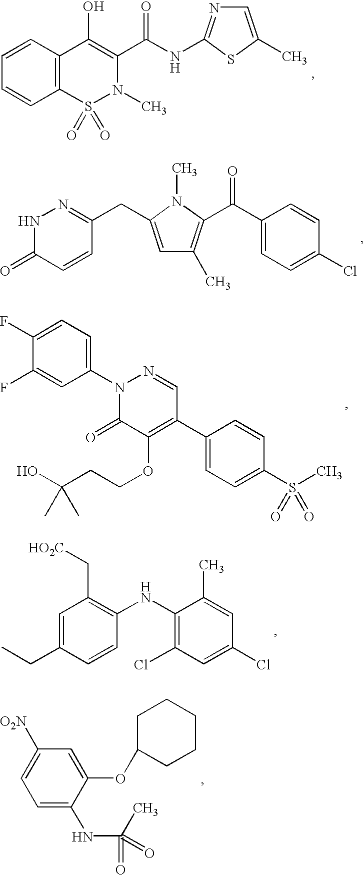

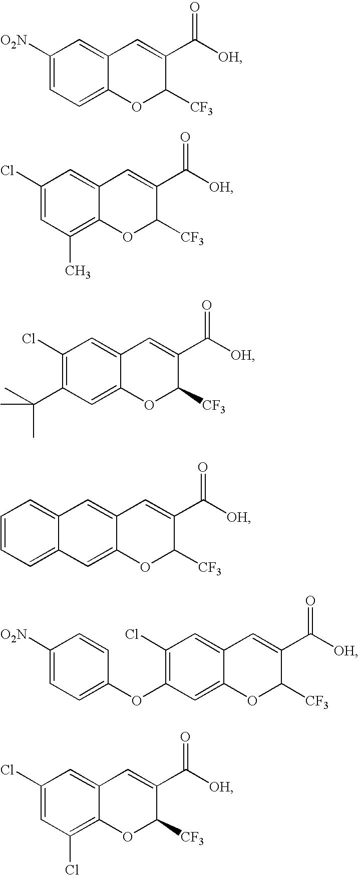

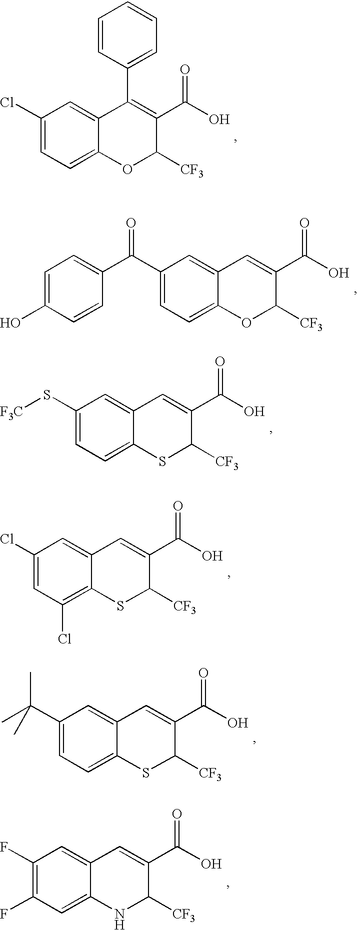

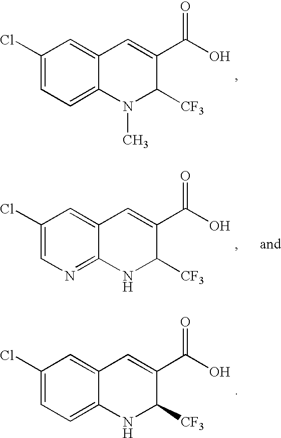

28. The method of claim 23, wherein the COX-2 inhibitor is selected from 6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphtho[2,1-b]pyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino] sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
6-chloro-2-trifluoromethyl-2H-1-benzothiopyran-3-carboxylic acid;
3-[(3-Chloro-phenyl)-(4-methanesulfonyl-phenyl)-methylene]-dihydro-furan-2-one;
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanonc;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl] spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl] spiro[3.4]oct-6-ene;
5-(3-chloro-4-methoxyphenyl)-6-[4-(methylsulfonyl)phenyl] spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1, 1-dimethylcyclopenta-2,4-dien-3-yl]benzenesulfonamide;
5-(4-fluorophenyl)-6-[4-(methylsulfonyl)phenyl] spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-fluorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzene sulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-7-(1,1-dimethylethyl)-2-trifluoromethyl-2H-1-benzopyran-3-carboxylic acid;
6-chloro-8-methyl-2-trifluoromethyl-2H-1-benzopyran-3-carboxylic acid;
5,5-dimethyl-3-(3-fluorophenyl)-4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide;
4-[5-(2-fluoro-4-methoxyphenyl)-2-trifluoromethyl-4-oxazolyl]benzenesulfonamide;
[2-(2-chloro-6-fluoro-phenylamino)-5-methyl-phenyl]-acetic acid;
N-(4-Nitro-2-phenoxy-phenyl)-methanesulfonamide or nimesulide;
N-[6-(2,4-difluoro-phenoxy)-1-oxo-indan-5-yl]-methanesulfonamide or flosulide;
N-[6-(2,4-Difluoro-phenylsulfanyl)-1-oxo-1H-inden-5-yl]-methanesulfonamide, soldium salt;
N-[5-(4-fluoro-phenylsulfanyl)-thiophen-2-yl]-methanesulfonamide;
3-(3,4-Difluoro-phenoxy)-4-(4-methanesulfonyl-phenyl)-5-methyl-5-(2,2,2-trifluoro-ethyl)-5H-furan-2-one;
(5Z)-2-amino-5-[[3,5-bis(1,1-dimethylethyl)-4-hydroxyphenyl]methylene]-4(5H)-thiazolone or darbufelone;
N-[3-(formylamino)-4-oxo-6-phenoxy-4H-1-benzopyran-7-yl]-methanesulfonamide;
(6aR,10aR)-3-(1,1-dimethylheptyl)-6a,7,10,10a-tetrahydro-1-hydroxy-6,6-dimethyl-6H-dibenzo[b,d]pyran-9-carboxylic acid;
4-[[3,5-bis(1,1-dimethylethyl)-4-hydroxyphenyl]methylene]dihydro-2-methyl-2H-1,2-oxazin-3(4H)-one;
6-dioxo-9H-purin-8-yl-cinnamic acid (B-231);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;
[2-(2,4-dichloro-6-ethyl-3,5-dimethyl-phenylamino)-5-propyl-phenyl]-acetic acid; or
an isomer, a pharmaceutically acceptable salt, ester or prodrug thereof.

29. The method of claim 23, wherein the COX-2 inhibitor has the formula 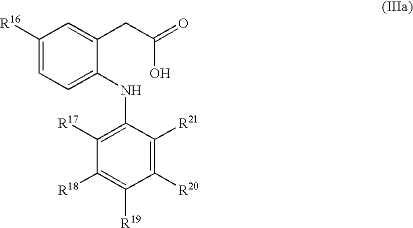
or an isomer, a pharmaceutically acceptable salt, ester, or prodrug thereof;
wherein
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

30. The method of claim 23, wherein the COX-2 inhibitor has the formula 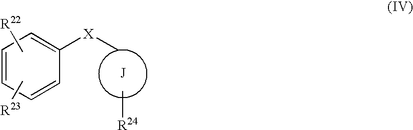
or an isomer, a pharmaceutically acceptable salt, an ester, or a prodrug thereof,
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

31. The method of claim 23, wherein the COX-2 inhibitor has the formula 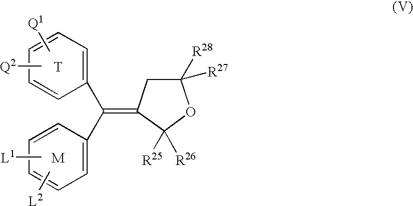
or an isomer, a pharmaceutically acceptable salt, an ester, or a prodrug thereof,
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

32. The method of claim 29, wherein R¹⁶ is ethyl.

33. The method of claim 29, where in R is methyl.

34. The method of claim 23, wherein the pharmaceutical composition comprises a permeation enhancer.

35. The method of claim 34, wherein the permeation enhancer comprises a compound selected from the group consisting of ethanol, isopropanol, 1,3-butanediol, oleyl alcohol, thymol, menthol, carvone, carveol, citral, dihydrocarveol, dihydrocarvone, neumenthol, isopulegol, terpene-4-ol, menthone, pulegol, camphor, geraniol, α-terpineol, linalol, carvacrol, t-anethole, and parecoxib.

36. The method of claim 35, wherein the permeation enhancer comprises a compound selected from the group of ethanol, isopropanol, 1,3-butanediol, oleyl alcohol, thymol, and paracoxib.

37. The method of claim 36, wherein the permeation enhancer comprises paracoxib.

38. The method of claim 23, wherein the selective COX-2 inhibitor is contained in the pharmaceutical composition in an amount of from 0.05-10 wt. %.

39. The method of claim 23, wherein the pharmaceutical composition comprises a glycol ether of the formula
R¹—O—((CH₂)ₘO)ₙ—R²
wherein R¹ and R² are independently hydrogen or C₁₋₆ alkyl, C₁₋₆ alkenyl, phenyl or benzyl group, with only one of R¹ and R² being hydrogen; m is an integer of 2 to 5 and n is an integer of 1 to 20.

40. The method of claim 23, wherein at least 25% by weight of the COX-2 inhibitor is in the form of nanoparticles having a particle size from about 450 to about 900 nm.

41. The method of claim 40, wherein at least 50% by weight of the COX-2 inhibitor is in the form of nanoparticles having a particle size from about 450 to about 900 nm.

42. The method of claim 41, wherein at least 75% by weight of the COX-2 inhibitor is in the form of nanoparticles having a particle size from about 450 to about 900 nm.

43. The method of claim 1, wherein the antiviral agent is a Podophyllin, a Nucleoside analoque, an Immunomodulator, an Antisense oligonucleotide, a Prophylactic vaccine, or a therapeutic vaccine.

44. The method of claim 43, wherein the antiviral agent is a Podophyllin, a Nucleoside analoque, or an Immunomodulator.

45. The method of claim 44, wherein antiviral agent is a Podophyllin.

46. The method of claim 45, wherein the Podophyllin is podofilox or podophyllin.

47. The method of claim 44, wherein the antiviral agent is a Nucleoside analoque.

48. The method of claim 47, wherein the Nucleoside analoque is selected from acyclovir, penciclovir, famciclovir, ganciclovir, BVDU, broavir, HPMPA, FIAC, FIAU, Cidofovir, Zidovudine, Zalcitabine, Didanosine, Lamivudine, Stavudine, vidarabine, ribavirin, and foscamet.

49. The method of claim 48, wherein the Nucleoside analoque is selected from vidarabine, ribavirin, and Cidofovir.

50. The method of claim 44, wherein the antiviral agent is an Immunomodulator.

51. The method of claim 50, wherein the Immunomodulator is Imiquimod.

52. The method of claim 23, wherein the antiviral agent is a Podophyllin, a Nucleoside analoque, an Immunomodulator, an Antisense oligonucleotide, a Prophylactic vaccine, or a therapeutic vaccine.

53. The method of claim 52, wherein the antiviral agent is a Podophyllin, a Nucleoside analoque, or an Immunomodulator.

54. The method of claim 53, wherein antiviral agent is a Podophyllin.

55. The method of claim 54, wherein the Podophyllin is podofilox or podophyllin.

56. The method of claim 53, wherein the antiviral agent is a Nucleoside analoque.

57. The method of claim 56, wherein the Nucleoside analoque is selected from acyclovir, penciclovir, famciclovir, ganciclovir, BVDU, broavir, HPMPA, FIAC, FIAU, Cidofovir, Zidovudine, Zalcitabine, Didanosine, Lamivudine, Stavudine, vidarabine, ribavirin, and foscarnet.

58. The method of claim 57, wherein the Nucleoside analoque is selected from vidarabine, ribavirin, and Cidofovir.

59. The method of claim 53, wherein the antiviral agent is an Immunomodulator.

60. The method of claim 59, wherein the Immunomodulator is Imiquimod.

61. A pharmaceutical composition, comprising a therapeutically effective amount of a COX-2 inhibitor or a pharmaceutically acceptable salt thereof and a therapeutically effective amount of an antiviral agent or a pharmaceutically acceptable salt thereof.

62. The pharmaceutical composition of claim 61, further comprising a permeation enhancer.

63. The pharmaceutical composition of claim 61, wherein the COX-2 inhibitor is a compound having the structure of Formula III 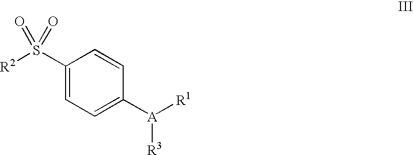
wherein A is a substituent selected from partially unsaturated or unsaturated heterocyclyl and partially unsaturated or unsaturated carbocyclic rings;
wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is methyl or amino; and
wherein R³ is a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

64. The pharmaceutical composition of claim 63, wherein the COX-2 inhibitor compound is celecoxib (A-21), valdecoxib (A-22), deracoxib (A-23), rofecoxib (A-24), etoricoxib (A-25), JTE-522 (A-26), or parecoxib (A-27).

65. The pharmaceutical composition of claim 63, wherein the COX-2 inhibitor is at least one member selected from the group consisting of celecoxib, valdecoxib and parecoxib.

66. The pharmaceutical composition of claim 61, wherein the COX-2 inhibitor is a compound selected from the group consisting of

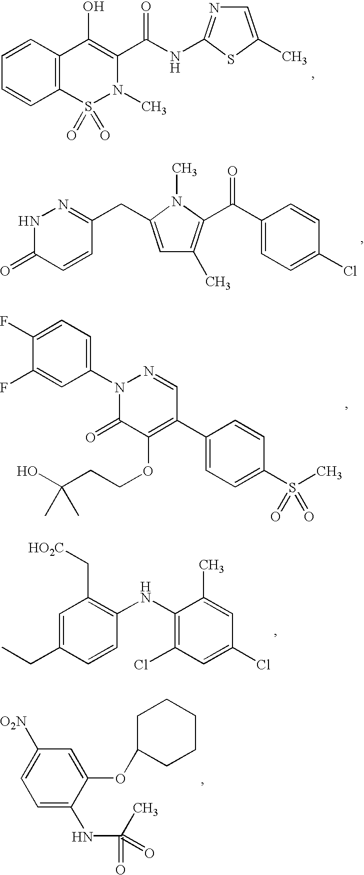

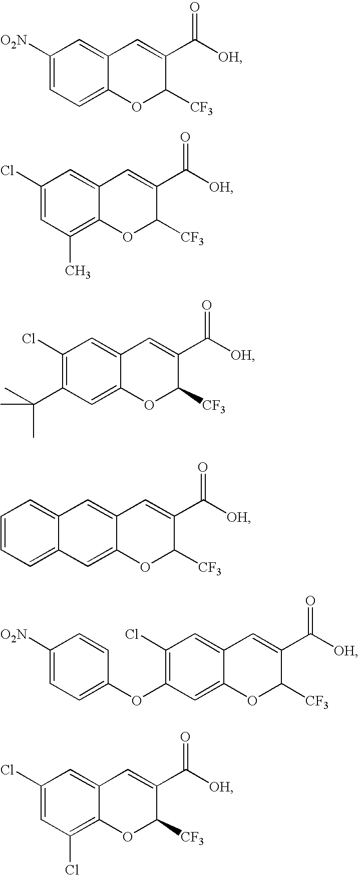

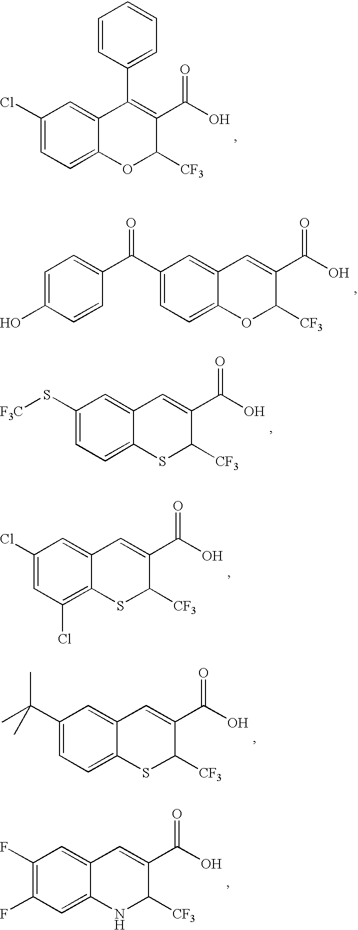

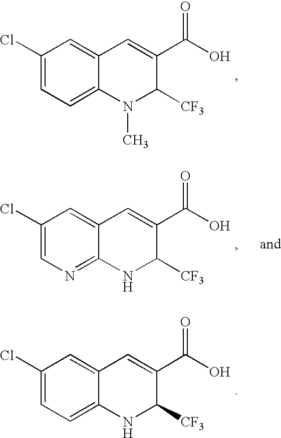

67. The pharmaceutical composition of claim 61, wherein the COX-2 inhibitor is selected from 6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphtho[2,1-b]pyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino] sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
6-chloro-2-trifluoromethyl-2H-1-benzothiopyran-3-carboxylic acid;
3-[(3-Chloro-phenyl)-(4-methanesulfonyl-phenyl)-methylene]-dihydro-furan-2-one;
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl] spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyll spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl) 1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-fluorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-] H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-7-(1, 1-dimethylethyl)-2-trifluoromethyl-2H-1-benzopyran-3-carboxylic acid;
6-chloro-8-methyl-2-trifluoromethyl-2H-1-benzopyran-3-carboxylic acid;
5,5-dimethyl-3-(3-fluorophenyl)-4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide;
4-[5-(2-fluoro-4-methoxyphenyl)-2-trifluoromethyl-4-oxazolyl]benzenesulfonamide;
[2-(2-chloro-6-fluoro-phenylamino)-5-methyl-phenyl]-acetic acid;
N-(4-Nitro-2-phenoxy-phenyl)-methanesulfonamide or nimesulide;
N-[6-(2,4-difluoro-phenoxy)-1-oxo-indan-5-yl]-methanesulfonamide or flosulide;
N-[6-(2,4-Difluoro-phenylsulfanyl)-1-oxo-1H-inden-5-yl]-methanesulfonamide, soldium salt;
N-[5-(4-fluoro-phenylsulfanyl)-thiophen-2-yl]-methanesulfonamide;
3-(3,4-Difluoro-phenoxy)-4-(4-methanesulfonyl-phenyl)-5-methyl-5-(2,2,2-trifluoro-ethyl)-5H-furan-2-one;
(5Z)-2-amino-5-[[3,5-bis(1,1-dimethylethyl)-4-hydroxyphenyl]methylene]-4(5H)-thiazolone or darbufelone;
N-[3-(formylamino)-4-oxo-6-phenoxy-4H-1-benzopyran-7-yl]-methanesulfonamide;
(6aR,10aR)-3-(1,1-dimethylheptyl)-6a,7,10,10a-tetrahydro-1-hydroxy-6,6-dimethyl-6H-dibenzo[b,d]pyran-9-carboxylic acid;
4-[[3,5-bis(1,1-dimethylethyl)-4-hydroxyphenyl]methylene]dihydro-2-methyl-2H-1,2-oxazin-3(4H)-one;
6-dioxo-9H-purin-8-yl-cinnamic acid (B-231);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;
[2-(2,4-dichloro-6-ethyl-3,5-dimethyl-phenylamino)-5-propyl-phenyl]-acetic acid; or
an isomer, a pharmaceutically acceptable salt, ester or prodrug thereof.

68. The pharmaceutical composition of claim 61, wherein the COX-2 inhibitor has the formula 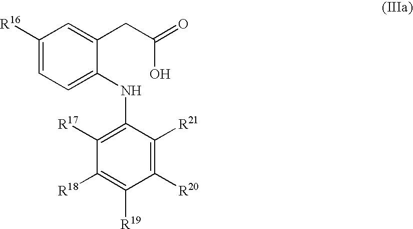
or an isomer, a pharmaceutically acceptable salt, ester, or prodrug thereof,
wherein
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

69. The pharmaceutical composition of claim 61, wherein the COX-2 inhibitor has the formula 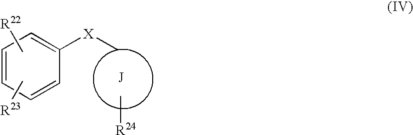
or an isomer, a pharmaceutically acceptable salt, an ester, or a prodrug thereof,
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

70. The pharmaceutical composition of claim 61, wherein the COX-2 inhibitor has the formula 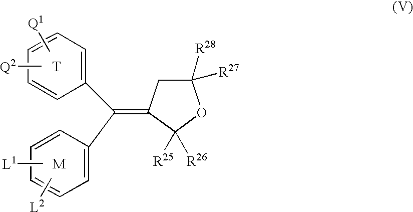
or an isomer, a pharmaceutically acceptable salt, an ester, or a prodrug thereof,
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

71. The pharmaceutical composition of claim 62, wherein the permeation enhancer comprises a compound selected from the group consisting of ethanol, isopropanol, 1,3-butanediol, oleyl alcohol, thyrnol, menthol, carvone, carveol, citral, dihydrocarveol, dihydrocarvone, neumenthol, isopulegol, terpene-4-ol, menthone, pulegol, camphor, geraniol, α-terpineol, linalol, carvacrol, t-anethole, and parecoxib.

72. The pharmaceutical composition of claim 71, wherein the permeation enhancer comprises a compound selected from the group of ethanol, isopropanol, 1,3-butanediol, oleyl alcohol, thymol, and paracoxib.

73. The pharmaceutical composition of claim 72, wherein the permeation enhancer comprises paracoxib.

74. The pharmaceutical composition of claim 61, wherein the COX-2 inhibitor is contained in the pharmaceutical composition in an amount of from about 0.05 to about 10 wt. %.

75. The pharmaceutical composition of claim 61, wherein the antiviral agent is contained in the pharmaceutical composition in an amount of from about 0.05 to about 10 wt. %.

76. The pharmaceutical composition of claim 61, further comprising a glycol ether of the formula
R¹—O—((CH₂)ₘO)ₙ—R²
wherein R¹ and R² are independently hydrogen or C₁₋₆ alkyl, C₁₋₆ alkenyl, phenyl or benzyl group, with only one of R¹ and R² being hydrogen; m is an integer of 2 to 5 and n is an integer of 1 to 20.

77. The pharmaceutical composition of claim 61, wherein the antiviral agent is a Podophyllin, a Nucleoside analoque, an Immunomodulator, an Antisense oligonucleotide, a Prophylactic vaccine, or a therapeutic vaccine.

78. The pharmaceutical composition of claim 77, wherein the antiviral agent is a Podophyllin, a a Nucleoside analoque, or an Immunomodulator.

79. The pharmaceutical composition of claim 78, wherein antiviral agent is a Podophyllin.

80. The pharmaceutical composition of claim 79, wherein the Podophyllin is podofilox or podophyllin.

81. The pharmaceutical composition of claim 78, wherein the antiviral agent is a Nucleoside analoque.

82. The pharmaceutical composition of claim 81, wherein the Nucleoside analoque is selected from acyclovir, penciclovir, famciclovir, ganciclovir, BVDU, broavir, HPMPA, FIAC, FIAU, Cidofovir, Zidovudine, Zalcitabine, Didanosine, Lamivudine, Stavudine, vidarabine, ribavirin, and foscarnet.

83. The pharmaceutical composition of claim 82, wherein the Nucleoside analoque is selected from vidarabine, ribavirin, and Cidofovir.

84. The pharmaceutical composition of claim 78, wherein the antiviral agent is an Immunomodulator.

85. The pharmaceutical composition of claim 84, wherein the Immunomodulator is Imiquimod.
